(19) United States
(12) Patent Application Publication
(10) Pub. No.: US 2025/0388530 A1
(43) Pub. Date: Dec. 25, 2025

(54) ALKYLAMINE-BASED IONIZABLE GLYCEROL OLEATE COMPOUNDS

(71) Applicant: THE BOARD OF TRUSTEES OF THE UNIVERSITY OF ILLINOIS, Urbana, IL (US)

(72) Inventors: Cecilia Das Neves Barbosa LEAL, Champaign, IL (US); Zhengzhong TAN, Savoy, IL (US); Lining ZHENG, Savoy, IL (US); Max BALIGA, Urbana, IL (US)

(73) Assignee: THE BOARD OF TRUSTEES OF THE UNIVERSITY OF ILLINOIS, Urbana, IL (US)

(21) Appl. No.: 19/249,389

(22) Filed: Jun. 25, 2025

Related U.S. Application Data
(60) Provisional application No. 63/663,781, filed on Jun. 25, 2024.

(51) Int. Cl. C07C 229/30; A61K 9/51; A61K 48/00; C12N 15/88

(52) U.S. Cl. CPC C07C 229/30; A61K 9/5123; A61K 48/0033; C12N 15/88

(57) ABSTRACT

RNA therapeutics have the potential to resolve a myriad of genetic diseases. Lipid nanoparticles (LNPs) are among the most successful RNA delivery systems. Expanding their use for the treatment of more genetic diseases hinges on our ability to continuously evolve the design of LNPs with high potency, cellular-specific targeting, and low side effects. Overcoming the difficulty of releasing cargo from endocytosed LNPs remains a significant hurdle. We investigated the fundamental properties of nonviral RNA nanoparticles pertaining to the activation of topological transformations of endosomal membranes and RNA translocation into the cytosol. We showed that, beyond composition, LNP fusogenicity can be prescribed by designing LNP nanostructures that lower the energetic cost of fusion and fusion-pore formation with a target membrane. The inclusion of structurally active lipids leads to enhanced LNP endosomal fusion, fast evasion of endosomal entrapment, and efficacious RNA delivery. For example, a compound of formula I:

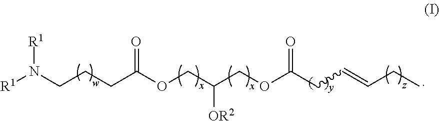

### RELATED APPLICATIONS

This application claims priority under 35 U.S.C. § 119 (e) to U.S. Provisional Patent Application No. 63/663,781, filed Jun. 25, 2024, which is incorporated herein by reference.

### GOVERNMENT SUPPORT

This invention was made with government support under R01GM143723 awarded by the National Institutes of Health. The United States Government has certain rights in the invention.

### BACKGROUND OF THE INVENTION

RNA therapeutics has gained wide attention due to its power to regulate the expression of disease-related genes that drive a myriad of human diseases, including cancer, neurodegeneration, and metabolic disorders, among others. Delivery vectors play an important role in the development of such therapeutics in preventing RNA degradation and successfully deliver RNA to target cells. Compared to viral vectors, nonviral systems have the advantage of being less immunogenic and easier to manufacture. Among the different materials used for nonviral delivery, lipid nanoparticles (LNPs) have been particularly successful, as many LNP-RNA formulations are clinically available or have advanced to clinical trials. The first Food and Drug Administration (FDA)-approved small interfering RNA (siRNA) treatment, patisiran, utilized LNPs for delivery. The effectiveness of LNPs has been further manifested by the development of lipid-mRNA (messenger RNA) vaccines for coronavirus disease 2019 (COVID-19).

Despite the astonishing progress LNP delivery achieved, many barriers are still present in realizing the full potential of LNP-RNA systems. While most LNP systems are efficiently taken up by the cell via endocytosis, they often remain trapped in endosomal compartments and degrade through the endosome-lysosome acidification pathway. One of the main bottlenecks of LNP-based RNA delivery is poor endosomal escape. Viral delivery systems have membrane proteins that undergo a conformation change allowing them to easily fuse with the plasma or endosomal membranes to release their payload. Previous studies have shown that for LNP mediated siRNA delivery, less than 2% of the siRNA can successfully escape the endosome and reach the cytosol. With low cytosolic delivery efficacy, much research has been done in an attempt to improve endosomal escape and transfection efficiency of LNPs by modifying lipid compositions. Sahay et al. (Nat. Biotechnol. 2013, 31, 653-658) have shown that LNPs containing cholesterol demonstrate improved delivery efficiencies, potentially caused by enhanced endosomal membrane fusion. Other ways of optimizing lipid compositions have also shown success, such as the inclusion of ionizable lipids. Ionizable lipids are able to stay neutral under physiological pH but become cationic under the acidic environment of endosomes, being able to electrostatically bind the endosomal membrane which may allow LNPs to destabilize it and facilitate escape. Siegwart's (Proc. Natl. Acad. Sci. U.S.A. 2021, 118, e2109256118) team has also shown that the lipid composition of LNPs can be used to target the particles to specific organs.

Accordingly, there is a need for improved lipid nanoparticles that can deliver their therapeutic cargo to cells more efficiently.

### SUMMARY

Glycerol monooleate (GMO) has been demonstrated by our team to be able to facilitate endosomal escape of lipid nanoparticles (LNPs) due to its unique negative Gaussian curvature. Current LNP formulation utilize ionizable lipids (SM-102, ALC-0315, etc.) that can be protonated in endosome environment to perform a similar task. Here, we want to combine the structural activity of GMO lipid and the charge-based activity of ionizable group by synthesizing the novel ionizable GMO lipid. With two mechanism of promoting endosomal escape, ionizable GMO have great potential in increasing endosomal escape and thus can be applied in LNP formulation for RNA delivery.

In LNP-based RNA delivery, the RNA payload needs to be delivered into cytosol in order to function. Most LNP-RNA formulations enter the cells via endocytosis and end up in endosome. LNPs need to escape the endosome to deliver the RNA payload to cytosol, before the endosome fuses with lysosome and thus degrading all the nanoparticles trapped inside. One of the major bottlenecks in LNP-RNA formulation is that the percentage of nanoparticles that escapes endosome is very low, roughly 2%. Current FDA approved LNP formulations for RNA therapeutics contains ionizable lipids, such as 9-Heptadecanyl 8-{(2-hydroxyethyl)[6-oxo-6-(undecyloxy) hexyl]amino}octanoate (SM-102), [(4-Hydroxybutyl)azanediyl]di(hexane-6,1-diyl) bis(2-hexyldecanoate) (ALC-0315), (6Z,9Z,28Z,31Z)-Heptatriaconta-6,9,28,31-tetraen-19-yl 4-(dimethylamino)butanoate (D-Lin-MC3-DMA). They contain tertiary amine that can be protonated in slightly acidic environment in endosome, and the positive charge helps to disrupt the endosome membrane and facilitates endosomal escape. We have demonstrated a completely different endosome disruption mechanism by glycerol monooleate (GMO) lipid, which forms membrane with negative Gaussian curvature. We prepared a novel lipid class that contains both tertiary amine group and the glycerol monooleate, termed dimethylamine-based ionizable glycerol monooleate derivatives (iGMOs), that can disrupt the endosome membrane by both the charge-based mechanism and the curvature-based mechanism, increasing the efficiency of endosomal disruption and thus increase the delivery efficiency of RNA delivery.

Our invention can be utilized in LNP formulation for RNA therapeutics delivery to replace current ionizable lipids to help the endosomal escape of the RNA payload through two mechanisms. RNA therapeutics have huge potentials in combating a variety of diseases, for example, the COVID-19 vaccines from Moderna and Pfizer as well as numerous LNP-based gene therapy candidates in clinical trials.

Accordingly, this disclosure provides a compound of formula I:

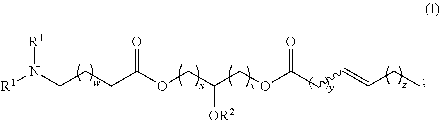

or a pharmaceutically acceptable salt thereof.
wherein
each R¹ is independently —(C₁-C₆)alkyl or H;
R² is H or —(C₁-C₆)alkyl;
w is 1-3 or 0;
each x is independently 1-3;
y is 7 or 1-10; and
z is 8 or 1-10.

Also, this disclosure also provides a lipid nanoparticle composition comprising one or more lipids and one or more of a compound described above.

Additionally, this disclosure provides a method for delivering biologically active cargo in a lipid nanoparticle into the cytosol of a cell comprising:
contacting a cell with a lipid nanoparticle wherein the lipid nanoparticle comprises biologically active cargo and a compound described above;
wherein the lipid nanoparticle (LNP) comprising Gaussian curvature forming compounds, such as a compound of formula I, enters the cell by endocytosis to form an endosome, and the LNP forms a fusion pore from within the endosome on its membrane, wherein the compound is ionized in the endosome's acidic environment to facilitate enhanced fusogenic disruption of the endosome's membrane and escape of the lipid nanoparticle and its cargo from the endosome, thereby delivering biologically active cargo of the escaped lipid nanoparticle into the cell's cytosol.

The invention provides novel compounds of formula I and formulas II, IIA, IIB, intermediates for the synthesis of compounds of formula I and formulas II, IIA, IIB, as well as methods of preparing compounds of formula I and formulas II, IIA, IIB,. The invention also provides compounds of formula I and formulas II, IIA, IIB, that are useful as intermediates for the synthesis of other useful compounds. The invention provides for the use of compounds of formula I and formulas II, IIA, IIB, for the manufacture of medicaments useful for the treatment of viral infections in a mammal, such as a human.

The invention provides for the use of the compositions described herein for use in medical therapy. The medical therapy can treat viral infections, for example, COVID 19 or flu, or to treat a cancer or a genetic disorder. The invention also provides for the use of a composition as described herein for the manufacture of a medicament to treat an infection in a mammal, for example, a viral infection in a human, or to treat a cancer or a genetic disorder. The medicament can include a pharmaceutically acceptable diluent, excipient, or carrier.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. Mass spectrum of compound iGMO.

FIG. 2. Graph showing in vitro mRNA delivery efficiency of LNP.

FIG. 3A-B. Graphs showing iGMO LNP +/−mRNA size characterization by nanoparticle tracking analysis (NTA). (A) −mRNA, average size: 104 nm; averaged FTLA concentration/size for experiment: 4_minusmRNA_1000×_diluted 2024 Jan. 24 15-33-10. Error bars indicate +/−1 standard error over the mean. (B) +mRNA, average size: 148 nm; averaged FTLA concentration/size for experiment: 4_plusmRNA_1000×_diluted 2024 Jan. 24 13-17-30 2024 Jan. 24 13-54-29 2024 Jan. 24 14-38-49 2024 Jan. 24 15-16-59. Error bars indicate +/−1 standard error over the mean.

FIG. 4. iGMO LNP +mRNA formulation structural characterization by Cryogenic electron microscopy (Cryo-EM). Image showing small LNP (about 50 nm) loaded with mRNA.

FIG. 5. Small angle X-ray scattering (SAXS)-Bulk hydrated lipids. Mixtures of GMO and iGMO. Pure GMO is diamond inverse cubic. Pure iGMO forms the primitive cubic phase. Mixtures show inverse hexagonal and mixed inverse hexagonal and primitive cubic phases. Preference for cubic and hexagonal phases is retained with modification of GMO headgroup to form iGMO. Cubic and hexagonal phases are associated with increased transfection efficiency

FIG. 6A-B. SAXS-bulk hydrated lipids. pH-dependent structural transitions, particularly around suspected iGMO pKa. Inverse hexagonal and cubic phases. Addition of DOTAP favors lamellar phases. pKa of the iGMO amine expected to be ˜6.5

FIG. 7A-B. SAXS-bulk hydrated lipids. pH-dependent structural transformations, particularly around iGMO pKa. Inverse hexagonal and cubic phase. Addition of GMO favors cubic phases across pH. pKa of the iGMO amine expected to be ˜6.5

FIG. 8. SAXS-bulk hydrated lipids: DSPC favors lamellar phases. POPC favors inverse hexagonal phases. Future formulations could replace DSPC with POPC.

FIG. 9A-B. SAXS—bulk lipids+mRNA. Some evidence for an inverse hexagonal to lamellar phase transition as pH decreases. Some mixed-phase lamellar and inverse hexagonal phases

FIG. 10A-C. Encapsulation efficiency—iGMO-based LNPs. A. iGG vaccine formulation LNPs. B. iGD and iGGD vaccine formulation LNPs. Addition of DOTAP helps to encapsulate more mRNA. Further addition of GMO reduces encapsulation efficiency and encapsulated mRNA concentration. Different trends of mFLuc and mEGFP encapsulation over the various formulations-mFLuc mRNA is twice as long as mEGFP. C. iGMO-based LNPs. Changing helper lipid identity drastically alters encapsulation efficiency. DOPE and POPC are good candidates for helper lipids based on the cubic structures observed in X-ray scattering.

FIG. 11A-C. In vitro transfection efficiency. Formulations perform differently with mFLuc vs. mEGFP.

FIG. 12. In vitro transfection efficiency+Live/Dead. Transfection is consistent with previous mEGFP flow experiment. Small increase in dead cell rate with 300 ng mEGFP iGD LNPs. FIG. 13A-B. CryoEM—iG vaccine formulation LNPs. Electron beam damaged areas (circles) indicate the likely presence of mRNA in the denser phase. Large aggregates.

FIG. 14A-B. CryoEM—iGG vaccine formulation LNPs. Electron beam damaged areas (circles) indicate the likely presence of mRNA in the denser phases. Core-shell structure. Isolated, smaller nanoparticles.

FIG. 15. CryoEM—iGG vaccine formulation LNPs. Large range of morphologies: multivesicular, encased and free dense cores, only bilayers, range of sizes.

FIG. 16. CryoEM—iGD vaccine formulation LNPs. Small particles, encased and free dense cores.

FIG. 17. CryoEM—iGGD vaccine formulation LNPs. Small particles, encased and free dense cores.

FIG. 18. SAXS: iGMO/Chol/DOPE vs. pH. Inverse cubic phases. Transitions from mostly primitive to diamond at pH=7. Weak primitive reflections present at several pHs (see arrows). Compare to iGMO/Chol/DSPC and iGMO/Chol/POPC vs. pH.

### DETAILED DESCRIPTION

RNA therapeutics have the potential to resolve a myriad of genetic diseases. Lipid nanoparticles (LNPs) are among the most successful RNA delivery systems. Expanding their use for the treatment of more genetic diseases hinges on our ability to continuously evolve the design of LNPs with high potency, cellular-specific targeting, and low side effects. Overcoming the difficulty of releasing cargo from endocytosed LNPs remains a significant hurdle. Here, we investigate the fundamental properties of nonviral RNA nanoparticles pertaining to the activation of topological transformations of endosomal membranes and RNA translocation into the cytosol. We show that, beyond composition, LNP fusogenicity can be prescribed by designing LNP nanostructures that lower the energetic cost of fusion and fusion-pore formation with a target membrane. The inclusion of structurally active lipids leads to enhanced LNP endosomal fusion, fast evasion of endosomal entrapment, and efficacious RNA delivery. For example, conserving the lipid make-up, RNA-LNPs having cuboplex nanostructures are significantly more efficacious at endosomal escape than traditional lipoplex constructs.

Additional information and data supporting the invention can be found in the following publication by the inventors: L. Zheng et al, PNAS 2023, 120 (27), e2301067120 and its Supporting Information, which are incorporated herein by reference in their entirety.

### Definitions.

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five, or one to four, for example if the phenyl ring is disubstituted.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number1” to “number2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number10”, it implies a continuous range that includes whole numbers and fractional numbers less than number10, as discussed above. Similarly, if the variable disclosed is a number greater than “number10”, it implies a continuous range that includes whole numbers and fractional numbers greater than number10. These ranges can be modified by the term “about”, whose meaning has been described above.

The recitation of a), b), c), . . . or i), ii), iii), or the like in a list of components or steps do not confer any particular order unless explicitly stated.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

Alternatively, the terms “effective amount” or “therapeutically effective amount,” as used herein, refer to a sufficient amount of an agent or a composition or combination of compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration of the compositions, the type or extent of supplemental therapy used, ongoing disease process and type of treatment desired (e.g., aggressive vs. conventional treatment).

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

As used herein, “subject” or “patient” means an individual having symptoms of, or at risk for, a disease or other malignancy. A patient may be human or non-human and may include, for example, animal strains or species used as “model systems” for research purposes, such a mouse model as described herein. Likewise, the patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods provided herein, the mammal is a human.

As used herein, the terms “providing”, “administering,” “introducing,” are used interchangeably herein and refer to the placement of a compound of the disclosure into a subject by a method or route that results in at least partial localization of the compound to a desired site. The compound can be administered by any appropriate route that results in delivery to a desired location in the subject.

The compound and compositions described herein may be administered with additional compositions to prolong stability and activity of the compositions, or in combination with other therapeutic drugs.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation or limitations which is not specifically disclosed herein.

This disclosure provides methods of making the compounds and compositions of the invention. The compounds and compositions can be prepared by any of the applicable techniques described herein, optionally in combination with standard techniques of organic synthesis. Many techniques such as etherification and esterification are well known in the art. However, many of these techniques are elaborated in Compendium of Organic Synthetic Methods (John Wiley & Sons, New York), Vol. 1, Ian T. Harrison and Shuyen Harrison, 1971; Vol. 2, Ian T. Harrison and Shuyen Harrison, 1974; Vol. 3, Louis S. Hegedus and Leroy Wade, 1977; Vol. 4, Leroy G. Wade, Jr., 1980; Vol. 5, Leroy G. Wade, Jr., 1984; and Vol. 6; as well as standard organic reference texts such as March's Advanced Organic Chemistry: Reactions, Mechanisms, and Structure, 5th Ed., by M. B. Smith and J. March (John Wiley & Sons, New York, 2001); Comprehensive Organic Synthesis. Selectivity, Strategy & Efficiency in Modern Organic Chemistry. In 9 Volumes, Barry M. Trost, Editor-in-Chief (Pergamon Press, New York, 1993 printing); Advanced Organic Chemistry, Part B: Reactions and Synthesis, Second Edition, Cary and Sundberg (1983); for heterocyclic synthesis see Hermanson, Greg T., Bioconjugate Techniques, Third Edition, Academic Press, 2013.

The formulas and compounds described herein can be modified using protecting groups. Suitable amino and carboxy protecting groups are known to those skilled in the art (see for example, Protecting Groups in Organic Synthesis, Second Edition, Greene, T. W., and Wuts, P. G. M., John Wiley & Sons, New York, and references cited therein; Philip J. Kocienski; Protecting Groups (Georg Thieme Verlag Stuttgart, New York, 1994), and references cited therein); and Comprehensive Organic Transformations, Larock, R. C., Second Edition, John Wiley & Sons, New York (1999), and referenced cited therein.

The term “halo” or “halide” refers to fluoro, chloro, bromo, or iodo. Similarly, the term “halogen” refers to fluorine, chlorine, bromine, and iodine.

The term “alkyl” refers to a branched or unbranched hydrocarbon having, for example, from 1-20 carbon atoms, and often 1-12, 1-10, 1-8, 1-6, or 1-4 carbon atoms; or for example, a range between 1-20 carbon atoms, such as 2-6, 3-6, 2-8, or 3-8 carbon atoms. As used herein, the term “alkyl” also encompasses a “cycloalkyl”, defined below. Examples include, but are not limited to, methyl, ethyl, 1-propyl, 2-propyl (iso-propyl), 1-butyl, 2-methyl-1-propyl (isobutyl), 2-butyl (sec-butyl), 2-methyl-2-propyl (t-butyl), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, hexyl, octyl, decyl, dodecyl, and the like. The alkyl can be unsubstituted or substituted, for example, with a substituent described below or otherwise described herein. The alkyl can also be optionally partially or fully unsaturated. As such, the recitation of an alkyl group can include an alkenyl group or an alkynyl group. The alkyl can be a monovalent hydrocarbon radical, as described and exemplified above, or it can be a divalent hydrocarbon radical (i.e., an alkylene).

An alkylene is an alkyl group having two free valences at a carbon atom or two different carbon atoms of a carbon chain. Similarly, alkenylene and alkynylene are respectively an alkene and an alkyne having two free valences at two different carbon atoms, or an alkenylene can have the two free valences on the same carbon.

The term “cycloalkyl” refers to cyclic alkyl groups of, for example, from 3 to 10 carbon atoms having a single cyclic ring or multiple condensed rings. Cycloalkyl groups include, by way of example, single ring structures such as cyclopropyl, cyclobutyl, cyclopentyl, cyclooctyl, and the like, or multiple ring structures such as adamantyl, and the like. The cycloalkyl can be unsubstituted or substituted. The cycloalkyl group can be monovalent or divalent and can be optionally substituted as described for alkyl groups. The cycloalkyl group can optionally include one or more cites of unsaturation, for example, the cycloalkyl group can include one or more carbon-carbon double bonds, such as, for example, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, and the like.

The term “heteroatom” refers to any atom in the periodic table that is not carbon or hydrogen. Typically, a heteroatom is O, S, N, P. The heteroatom may also be a halogen, metal or metalloid.

The term “heterocycloalkyl” or “heterocyclyl” refers to a saturated or partially saturated monocyclic, bicyclic, or polycyclic ring containing at least one heteroatom selected from nitrogen, sulfur, oxygen, preferably from 1 to 3 heteroatoms in at least one ring. Each ring is preferably from 3-to 10-membered, more preferably 4 to 7 membered. Examples of suitable heterocycloalkyl substituents include pyrrolidinyl, tetrahydrofuranyl, tetrahydrothiofuranyl, piperidinyl, piperazinyl, tetrahydropyranyl, morpholinyl, 1,3-diazapanyl, 1,4-diazapanyl, 1,4-oxazepanyl, and 1,4-oxathiapanyl. The group may be a terminal group or a bridging group.

As used herein, the term “substituted” or “substituent” is intended to indicate that one or more (for example, in various embodiments, 1-10; in other embodiments, 1-6; in some embodiments 1, 2, 3, 4, or 5; in certain embodiments, 1, 2, or 3; and in other embodiments, 1 or 2) hydrogens on the group indicated in the expression using “substituted” (or “substituent”) is replaced with a selection from the indicated group(s), or with a suitable group known to those of skill in the art, provided that the indicated atom's normal valency is not exceeded, and that the substitution results in a stable compound. Suitable indicated groups include, e.g., alkyl, alkenyl, alkynyl, alkoxy, haloalkyl, hydroxyalkyl, aryl, heteroaryl, heterocyclyl, cycloalkyl, alkanoyl, alkoxycarbonyl, amino, alkylamino, dialkylamino, carboxyalkyl, alkylthio, alkylsulfinyl, and alkylsulfonyl. Substituents of the indicated groups can be those recited in a specific list of substituents described herein, or as one of skill in the art would recognize, can be one or more substituents selected from alkyl, alkenyl, alkynyl, alkoxy, halo, haloalkyl, hydroxy, hydroxyalkyl, aryl, heteroaryl, heterocycle, cycloalkyl, alkanoyl, alkoxycarbonyl, amino, alkylamino, dialkylamino, trifluoromethylthio, difluoromethyl, acylamino, nitro, trifluoromethyl, trifluoromethoxy, carboxy, carboxyalkyl, keto, thioxo, alkylthio, alkylsulfinyl, alkylsulfonyl, and cyano. Suitable substituents of indicated groups can be bonded to a substituted carbon atom include F, Cl, Br, I, OR′, OC(O)N(R′)₂, CN, CF₃, OCF₃, R′, O, S, C(O), S(O), methylenedioxy, ethylenedioxy, N(R′)₂, SR', SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂NHC(O)R′, N(R′) N(R′) C(O)R′, N(R′) N(R′) C(O)OR′, N(R′) N(R′) CON(R′)₂, N(R′) SO₂R′, N(R′)SO₂N(R′)₂, N(R′)C(O)OR′, N(R′)C(O)R′, N(R′)C(S)R′, N(R′)C(O)N(R′)₂, N(R′)C(S) N(R′)₂, N(COR′)COR′, N(OR′)R′, C(═NH)N(R′)₂, C(O)N(OR′)R′, or C(═NOR′)R′ wherein R′ can be hydrogen or a carbon-based moiety (e.g., (C₁-C₆)alkyl), and wherein the carbon-based moiety can itself be further substituted. When a substituent is monovalent, such as, for example, F or Cl, it is bonded to the atom it is substituting by a single bond. When a substituent is divalent, such as O, it is bonded to the atom it is substituting by a double bond; for example, a carbon atom substituted with O forms a carbonyl group, C═O.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof, such as racemic mixtures, which form part of the present invention. Many organic compounds exist in optically active forms, i.e., they have the ability to rotate the plane of plane-polarized light. In describing an optically active compound, the prefixes D and L, or R and S. are used to denote the absolute configuration of the molecule about its chiral center(s). The prefixes d and l or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate (defined below), which may occur where there has been no stereoselection or stereospecificity in a chemical reaction or process.

The term “fusogenic disruption” refers to the fusion of cell membranes and formation of pores.

The term “helper lipid” refers to a class of lipid molecules that increase the particle stability and fluidity of lipid nanoparticles (LNP), for example, to improve particle stability, delivery efficacy, tolerability, and/or biodistribution.

### EMBODIMENTS OF THE TECHNOLOGY

1. A compound of formula I:

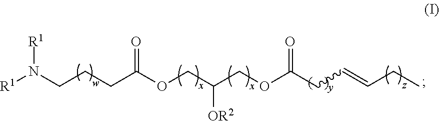

or a pharmaceutically acceptable salt thereof.
wherein
   each R¹ is independently —(C₁-C₆)alkyl or H;
   R² is H or —(C₁-C₆)alkyl;
   w is 0, 1, 2, or 3;
   each x is independently 1, 2, or 3;
   y is 1, 2, 3, 4, 5, 6, 7, 8, 9, or 10; and
   z is 1, 2, 3, 4, 5, 6, 7, 8, 9, or 10.
2. The compound of embodiment 1, wherein each R¹ is methyl.
3. The compound of embodiment 1 or 2, wherein R² is H.
4. The compound of any one of embodiments 1-3, wherein w is 1.
5. The compound of any one of embodiments 1-4, wherein each x is 1.
6. The compound of any one of embodiments 1-5, wherein each y is 7.
7. The compound of any one of embodiments 1-6, wherein each z is 8.
8. The compound of any one of embodiments 1-7, wherein the olefinic moiety of formula I has an E-configuration or Z-configuration.
9. The compound of any one of embodiments 1-8, wherein the compound is represented by formula II:

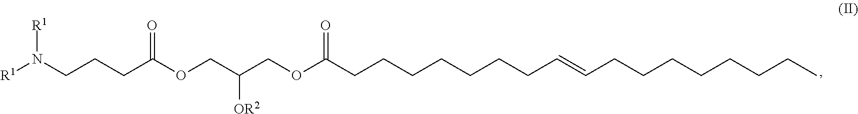

or a pharmaceutically acceptable salt thereof.

In some embodiments, the compound of formula II is represented by formula IIA or formula IIB:

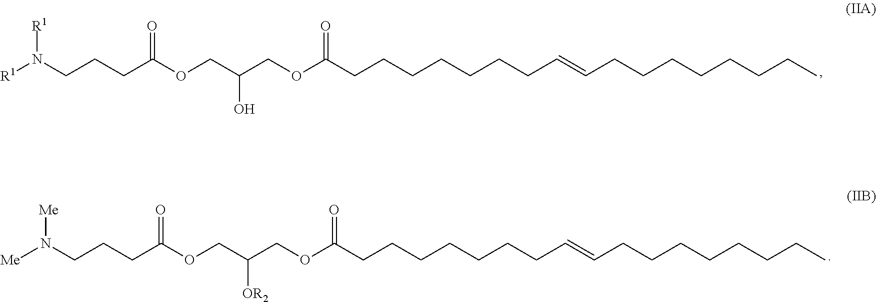

or a pharmaceutically acceptable salt thereof.
10. The compound of any one of embodiments 1-9, wherein the compound is iGMO:

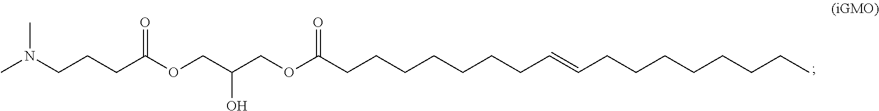 [SMILES: CCCCCCCC/C=C/CCCCCCCC(=O)OCC(O)COC(=O)CCCN(C)C]

or a pharmaceutically acceptable salt thereof.
11. A lipid nanoparticle (LNP) composition comprising one or more lipids and one or more of a compound of any one of embodiments 1-10.
12. The lipid nanoparticle composition of embodiment 11, further comprising RNA.
13. The lipid nanoparticle composition of embodiment 12, wherein the RNA is mRNA, siRNA, or both.
14. The lipid nanoparticle composition of any one of embodiments 11-13, further comprising a helper lipid, cholesterol, and a PEG-lipid conjugate.
15. The lipid nanoparticle composition of any one of embodiments 11-14, wherein the LNP composition comprises about 40 mol % to about 60 mol % iGMO, about 5 mol % to about 15 mol %

DSPC, about 25 mol % to about 45 mol % Cholesterol, and about 0.2 mol % to about 4 mol % DMPG-PEG2k. In some embodiments, the LNP composition comprises about 45 mol % to about 55 mol % iGMO, about 8 mol % to about 12 mol % DSPC, about 35 mol % to about 40 mol % Cholesterol, and about 0.5 mol % to about 2 mol % DMPG-PEG2k. In some embodiments, the content of GMO compounds can be a combination of iGMO with non-ionizable GMO.
16. The lipid nanoparticle composition of any one of embodiments 11-15, wherein the LNP composition comprises an LNP particles having an average size or average diameter of about 180 nm to about 300 nm, about 140 nm to about 220 nm, about 25 nm to about 75 nm, about 40 nm, about 50 nm, about 60 nm, about 70 nm, about 80 nm, about 90 nm, about 100 nm, about 125 nm, about 150 nm, about 175 nm, about 200 nm, about 225 nm, about 250 nm, or about 275 nm.
17. The lipid nanoparticle composition of any one of embodiments 11-16, wherein the composition comprises:
   about 45 mol % to about 55 mol % iGMO;
   about 33.5 mol % to about 43.5 mol % cholesterol;
   about 5 mol % to about 15 mol % 1,2-distearoyl-sn-glycero-3-phosphocholine (DSPC); and
   about 0.5 mole % to about 2.0 mole % 1,2-dimyristoyl-rac-glycero-3-methoxypolyethylene glycol-2000 (DMG-PEG2k).
18. The lipid nanoparticle composition of any one of embodiments 11-16, wherein the composition comprises:
   about 20 mol % to about 30 mol % iGMO;
   about 20 mol % to about 30 mol % glycerol monooleate (GMO);
   about 33.5 mol % to about 43.5 mol % cholesterol;
   about 5 mol % to about 15 mol % DSPC; and
   about 0.5 mole % to about 2.0 mole % DMG-PEG2k.
19. The lipid nanoparticle composition of any one of embodiments 11-16, wherein the composition comprises:
   about 20 mol % to about 30 mol % iGMO;
   about 20 mol % to about 30 mol % 1,2-dioleoyl-3-trimethylammonium propane (DOTAP);
   about 33.5 mol % to about 43.5 mol % cholesterol;
   about 5 mol % to about 15 mol % DSPC; and
   about 0.5 mole % to about 2.0 mole % DMG-PEG2k.
20. The lipid nanoparticle composition of any one of embodiments 11-16, wherein the composition comprises:
   about 20 mol % to about 30 mol % iGMO;
   about 10 mol % to about 20 mol % GMO;
   about 5 mol % to about 15 mol % DOTAP;
   about 33.5 mol % to about 43.5 mol % cholesterol;
   about 5 mol % to about 15 mol % DSPC; and
   about 0.5 mole % to about 2.0 mole % DMG-PEG2k.
21. The lipid nanoparticle composition of any one of embodiments 11-16, wherein the composition comprises:
   about 20 mol % to about 30 mol % iGMO;
   about 20 mol % to about 30 mol % GMO;
   about 33.5 mol % to about 43.5 mol % cholesterol;
   about 5 mol % to about 15 mol % POPC or DOPE; and
   about 0.5 mole % to about 2.0 mole % DMG-PEG2k.
22. A method for delivering biologically active cargo in a lipid nanoparticle into the cytosol of a cell comprising:
   contacting a cell with a lipid nanoparticle wherein the lipid nanoparticle comprises biologically active cargo and a compound of any one of embodiments 1-10; or
   contacting a cell with a lipid nanoparticle having a LNP composition of any one of embodiments 11-16 wherein the lipid nanoparticle comprises biologically active cargo such as mRNA, siRNA, or both, miRNA, or any other RNA/DNA combination;
   wherein the lipid nanoparticle (LNP) enters the cell by endocytosis to form an endosome and a fusion pore from within the endosome on the endosome's membrane, wherein the compound is ionized in the endosome's acidic environment to facilitate enhanced fusogenic disruption of the endosome's membrane and escape of the lipid nanoparticle from the endosome, thereby delivering biologically active cargo of the escaped lipid nanoparticle into the cell's cytosol or cytoplasm. In some embodiments the LNP and/or endosome has a negative Gaussian curvature on its membrane that is induced by the compounds described in any one of embodiments 1-10.
23. The method of embodiment 22, wherein the biologically active cargo is mRNA, siRNA, or both.
24. The method of embodiment 22 or 23, wherein the compound is 3- ((4-(dimethylamino) butanoyl)oxy)-2-hydroxypropyl (E)-octadec-9-enoate (iGMO).
25. The method of any one of embodiments 22-24, wherein the method treats a viral infection, cancer, or a genetic disorder, wherein the method can be applied to a specific target or specific organ such as lungs.
26. Use of a compound or composition according to any one of embodiments 1-21 for the manufacture of a virus vaccine for the treatment of a viral infection, or an immunotherapeutic for the treatment of a cancer; or for use in gene editing to treat a genetic disorder.

In some embodiments, the compositions described herein comprise inverse cubic phases, primitive cubic phases, hexagonal phases, inverse hexagonal phases, lamellar phases, or mixed phases.

### Lipid Nanoparticle Topology Regulates Endosomal Escape and Delivery of RNA to the Cytoplasm.

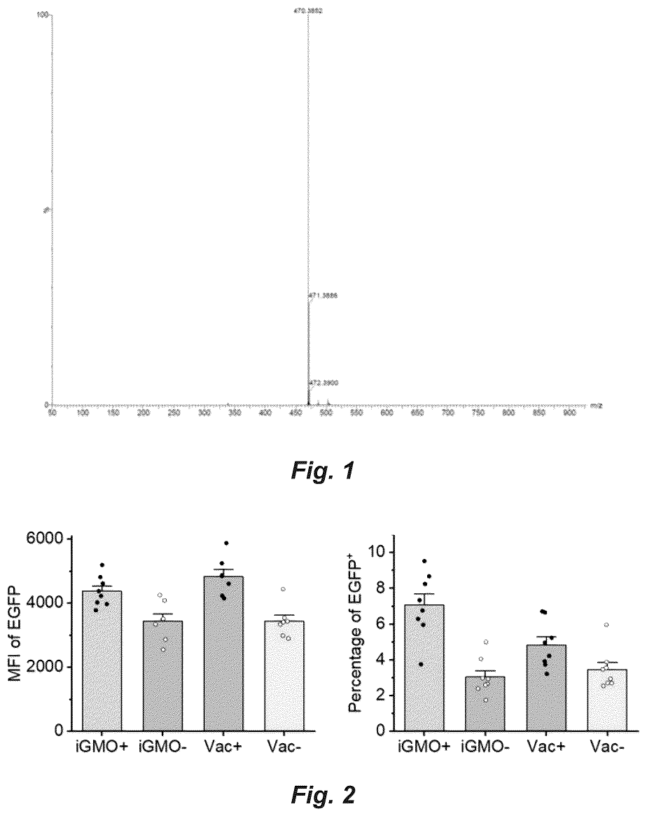

Compared to the extensive research done exploring different lipid formulations, the nanostructure of LNPs or the specific packing of lipids and nucleic acids into the LNP has not been fully investigated. However, we established structural activity as an LNP design principle. Due to their amphiphilic nature, lipid molecules can form a variety of self-assembled structures in aqueous environments (FIG. 1B, see Zheng et al.) that are conserved when encapsulating nucleic acids. This includes lamellar vesicles (L), inverse hexagonal (H_{II}), and bicontinuous cubic phases ( [symbol] _{II}) (34-44). The optimal mechanism to boost LNPs endosomal escape is to promote LNP-endosomal membrane fusion, a process that should take place in less than 30 s and be independent of endosome acidification or the debated “proton sponge” effect. The design of “fusogenic” LNPs has been exclusively attributed to tuning the molecular packing parameter of lipid molecules such that their spontaneous membrane curvature (Co) is negative. However, the elastic energy cost of membrane fusion or, more importantly, the development of the required fusion pore is controlled by a topological transformation that should mostly depend on the Gaussian moduli of membranes κ̅. The membrane elastic energy can be represented by the Helfrich equation,

$$E / A = \frac{1}{2} {κ ( J - {J}_{0} )}^{2} + \overline{κ} K ,$$

where κ is the bending modulus, J is the total/extrinsic curvature which equals the sum of the principle curvatures C₁+C₂, and C₀ is the spontaneous curvature. The second term comprises the

Gaussian curvature K=C₁C₂ and the Gaussian modulus κ̅. Taking into account the Gauss-Bonnet theorem and the Helfrich framework, one can calculate that the elastic energy cost of the topological transformation from two nested membrane vesicles (Einitial=8πκ⁻) to two fused vesicles (Efused=4πκ̅) to be ΔEfusion =−4πκ̅ (FIG. 1A, see Zheng et al.). It is immediately clear that modulating the Gaussian modulus κ̅ and Gaussian curvature K will significantly impact membrane fusion events and the formation of fusion pores through which RNA cargo can be delivered into the cytosol (FIG. 1C, see Zheng et al.). There are three main categories of reported LNP-nucleic acid nanostructures: L, H_{II}, and [symbol] _{II}. In the lamellar L phase, nucleic acids are sandwiched between lipid bilayers and are often referred to as “lipoplexes”. In H_{II}, nucleic acids are inserted in water tubes decorated by lipids, and in [symbol] _{II}, cuboplexes, siRNA has been shown to locate in the water nanochannel domains. Positively curved vesicular lamellar phases (L) have κ̅<0 but cubosomes or cuboplexes [cubosomes loaded with RNA], which are made of bicontinuous cubic phases, have intrinsically negative Gaussian curvature K and positive κ̅, and the activation energy for fusion with endosomal membranes and fusion pore formation should be the lowest. Siegel (Biophys. J. 1986, 49, 1171-1183) first suggested that bicontinuous cubic phases should be highly fusogenic, and we conjecture that [symbol] _{II} LNPs have a greater potential in fusing with the endosomal membrane and successfully release their cargo (FIG. 1C, see Zheng et al.). In addition, the bicontinuous nature of membranes can endow LNPs with gene/drug release properties sustained over longer periods of time which has been demonstrated for polymer-based systems by Scott's team (Nanoscale Horiz. 2019, 4, 258-272).

Here we show that, in addition to lipid molecular properties and composition, the structure of LNP-RNA complexes is a powerful design handle to boost the extent of endosomal-membrane fusion and concomitant efficacy of RNA cytoplasmic delivery.

### Results.

Formation of LNP-RNA Complexes of Precise Nanostructures. In our study, we judiciously select specific neutral lipid molecular systems and compositions to control the nanostructure of LNP-RNA complexes. Glycerol monooleate (GMO) is a neutral lipid approved by the FDA for in vivo use and is utilized as an adjuvant. GMO is well known to be stabilize into a variety of bicontinuous cubic phases that can be formulated into cubosome LNPs ( [symbol] _{II}) encapsulating siRNA which we termed cuboplexes. The cuboplex LNP formulation comprised GMO, a cationic lipid 1,2-dioleoyl-3-trimethylammonium propane (DOTAP) just enough to electrostatically bind RNA without raising toxicity as well as 1,2-dioleoyl-sn-glycero-3-phosphoethanolamine-N-[methoxy(polyethylene glycol)]-2000 (DOPE-PEG) to optimize the colloidal stability of the LNPs. Modulating the amount of neutral lipid GMO in the ternary GMO, DOTAP, and DOPE-PEG mixture results in precise tuning of different bicontinuous cubic structures of distinct space groups (Ia3d—gyroid, Im3m—primitive, and Pn3m—diamond) as well as the formation of hexagonal phases (H_{II}).

To form the traditional lamellar phase (L), GMO contents are low (<25 mol %), or the neutral lipid was switched to a phosphatidylcholine, 1,2-dioleoyl-sn-glycero-3-phosphocholine (DOPC). LNP-RNA complexes are formed with an optimal charge ratio of ρ=3, where charge ratio represents the number of positive charges from lipid molecules (n_{DOTAP}) over the number of negative charges from RNA (n_{NA}). When nucleic acid is added to the lipid systems, the nanostructures are mostly conserved, but there is a preference to adopt H_{II} phases as the 1D straight cylindrical water channels can accommodate long-chain nucleic acids.

FIG. 2 of Zheng et al. shows structural information of dilute LNP-RNA complexes (siRNA and mRNA) at different amounts of neutral GMO obtained by synchrotron small-angle X-ray scattering (SAXS). LNP-siRNA complexes with a GMO/DOTAP/DOPE-PEG (molar ratio 85/14/1) composition show four distinct diffraction peaks that indicate the presence of a bicontinuous cubic gyroid nanostructure ( [symbol] _{II}) coexisting with a 2D inverse hexagonal phase (H_{II}) (FIG. 2A, see Zheng et al.). The first two peaks show reciprocal lattice vectors q/(2π/a)=Gₕₖₗ/(2π/a)=(h²+k²+1²)^{1/2}=6, 8, which corresponds to the {211}, {220}planes of a Q_{II} gyroid phase. The first, third, and fourth peaks show reciprocal lattice vectors q/(4m/a3) =Ghk/(4π/a3)=(h²+hk+k²)^{1/2}=1, 3, and 4, corresponding to {10}, {11}, and {20}of the H_{II} phase. The lattice spacings are spec [symbol] =127 Å and a_{HII} =60 Å. LNP-mRNA complexes with the same lipid composition have a similar scattering profile and display H_{II}/ [symbol] _{II} phase coexistence with lattice spacings [symbol] =124 Å and a_{HII}=51 Å.

Due to the electrostatic interaction with negatively charged nucleic acids and the cationic lipid headgroups, nucleic acids are preferentially located in the water channels. We previously imaged using cryo-EM gold-tagged siRNA colocalized with the water domains of cuboplexes (ACS Nano 2018, 12, 9196-9205). Using the unit cell size of the cubic phase (a=127 Å), the diameter of the water channels can be estimated to be 33 Å which is just enough to tightly pack RNA (diameter of 22 Å with a hydration layer). This short-range electrostatic interaction will be dominant, and RNA is not expected to unbind and diffuse out of the cuboplex during organic solvent removal (by dialysis or evaporation). At intermediate GMO contents (FIG. 2B, see Zheng et al.), LNP-RNA complexes mostly adopt the H_{II} phase in coexistence with the L phase. The lattice spacings of the complexes are similar, with LNP-siRNA a_{HII}=63 Å and a_{L}=59 Å, and LNP-mRNA a_{HII}=103 Å and a_{L}=91 Å.At low GMO content (below 25 mol %), LNP-RNA complexes adopt the L phase (Fig. S1, see Zheng et al.). Switching GMO for a phosphatidylcholine neutral lipid always results in a L phase shown in FIG. 2C of Zheng et al. for DOPC/DOTAP/DOPE-PEG (molar ratio 85/14/1). The complexes show similar lattice parameters, with LNP-siRNA a_{L}=332 Å and LNP-mRNA [symbol] =334 Å.

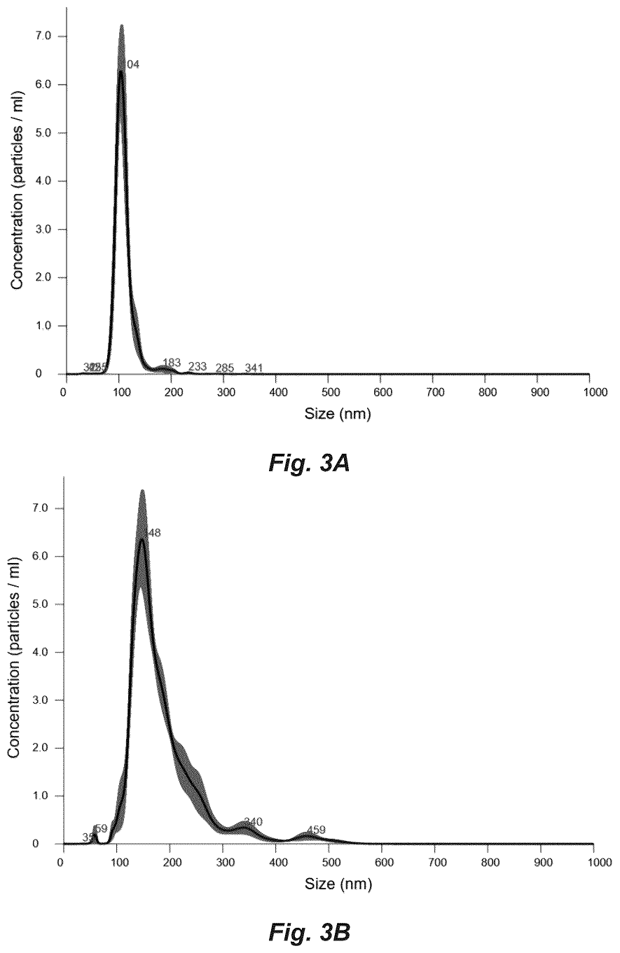

The structural diversity of LNP-RNA complexes is not only shown by SAXS but also manifested by cryogenic electron microscopy (cryo-EM). With the ability to image LNPs in their near-native hydrated state, cryo-EM has become an important technique to evaluate the structure of LNPs. FIG. 3 of Zheng et al. shows representative cryo-EM images of LNP-RNA complexes prepared by microfluidic methods with different nanostructures. FIG. 3A of Zheng et al. shows the lamellar structure of LNP-mRNA lipoplexes. In this “onion-like” structure, lipid domains intercalate with mRNA located in the water-layer domains. This is readily observable by fast Fourier transform (FFT, Inset) image analysis revealing periodic layers of electron density. FIG. 3B of Zheng et al. shows an LNP-siRNA complex of the H_{II} phase. In this nanostructure, siRNA aligns in the hexagonally packed water tubes that are decorated with lipids. The FFT image analysis clearly demonstrates the hexagonal symmetry of this LNP-siRNA complex.

A hybrid LNP-mRNA complex of both [symbol] _{II} and H_{II} is shown in FIG. 3C of Zheng et al. In this case, FFT image analysis of different regions of the particle reveals that this is a hybrid LNP system where both [symbol] _{II} and H_{II} are present. This is significant because SAXS data (FIG. 2A, see Zheng et al.) indicated the coexistence of these phases both for siRNA and mRNA systems. With SAXS being an average method, it was possible that both pure [symbol] _{II} and pure H_{II} LNPs coexisted in a test tube. The cryo-EM data indicate that, importantly, coexistence is present at a single-LNP level. FIG. 3D of Zheng et al. shows an LNP-siRNA cuboplex ( [symbol] _{II}) and respective FFT analysis where siRNA inserts in the water nanochannels. We also seen that while the preparation method does not significantly impact the nanostructure of the LNPs (Fig. S2, see Zheng et al.), top-down film hydration yields larger and more polydisperse systems compared to bottom-up nanoprecipitation in microfluidics (dialysis).

The modulation of neutral lipid amount and identity yields rich structural diversity of LNP-RNA complexes without disrupting their stability. LNP-RNA complexes remain colloidally stable regardless of the nanostructure having size distributions between 150 and 250 nm (Table 1).

TABLE 1
Sizes of LNP-RNA complexes measured
by nanoparticle tracking analysis.

| GMO/DOPC/DOTAP/DOPE-PEG | RNA type | Average size (nm) | |
|---|---|---|---|
| 85/0/14/1 | siRNA | 205.1 ± 2.5 | nm |
| 50/35/14/1 | siRNA | 190.5 ± 3.1 | nm |
| 25/60/14/1 | siRNA | 244.9 ± 4.5 | nm |
| 0/85/14/1 | siRNA | 265.2 ± 3.5 | nm |
| 85/0/14/1 | mRNA | 151.8 ± 10.0 | nm |
| 50/35/14/1 | mRNA | 163.4 ± 8.3 | nm |
| 25/60/14/1 | mRNA | 153.9 ± 4.5 | nm |
| 0/85/14/1 | mRNA | 200.8 ± 2.1 | nm |

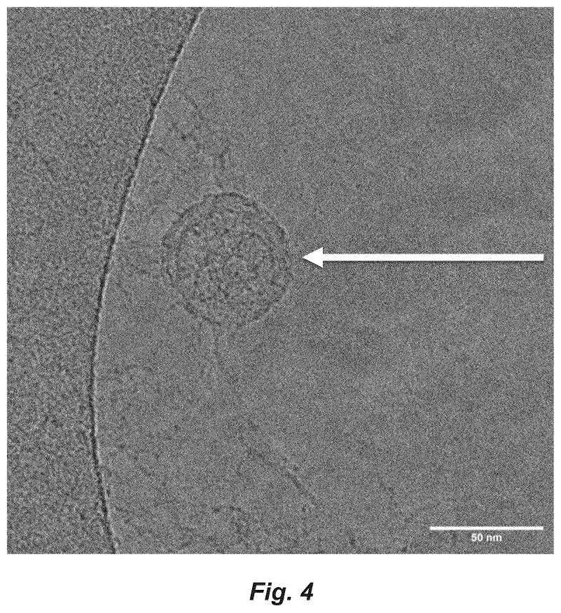

Fusion of LNP-RNA Complexes with Endosomes is Regulated by LNP Nanostructure. The interaction between endosomes and LNP-RNA complexes of different nanostructures in vitro was explored using confocal laser scanning microscopy (CLSM), fluorescence resonance energy transfer (FRET) assays, and live-cell self-quenching CLSM experiments (FIG. 4, see Zheng et al.). FIG. 4A of Zheng et al. shows CLSM images of isolated endosomes incubated with pure LNPs (no RNA) having the Z (liposome) and [symbol] _{II} (cubosomes) nanostructures. Endosomes were isolated by standard spin-column techniques from epithelial human cervix cancer (HeLa) cells and fluorescently labeled by incubating in 20 μM Nile Red. The LNPs were tagged with 0.1% 1,1′-dioctadecyl-3,3,3′,3′-tetramethylindodicarbocyanine (DiD). LNPs do not aggregate significantly on their own, but when incubated with endosomes, LNP-endosome fusion and aggregation are readily visible by colocalization of DiD and Nile Red fluorescence in both systems but is significantly more pronounced for [symbol] _{II} LNPs.

After observing that [symbol] _{II} LNPs bind with isolated endosomes more favorably, we conducted an FRET assay to evaluate the fusion process extent between endosomes and RNA-loaded LNPs of different nanostructures. Isolated endosomes were colabeled with 1,1′-dioctadecyl-3,3,3′,3′-tetramethylindocarbocyanine perchlorate (DiI) and 3,3′-dioctadecyloxacarbocyanine perchlorate (DiO), with DiO acting as a donor fluorophore and Dil acting as an acceptor fluorophore. The proximity of the dye molecules enables FRET to occur, leading to enhanced acceptor (DiI) fluorescence. If endosomes fuse with another membrane, the dye molecules diffuse, increasing the distance between each other and FRET occurrences diminish. Reduced FRET allows donor (DiO) fluorescence to recover. Thus, the extent of membrane fusion can be evaluated by the increase in DiO fluorescence. To see how LNP structure influences LNP-endosome membrane fusion, we prepared LNP-siRNA complexes with the same DOTAP molar fractions (Φ_{DOTAP}=0.14) but different GMO molar fractions. At Φ_{GMO}=0.85, RNA-loaded LNPs display the [symbol] _{II} structure (in coexistence with H_{II}) termed cuboplexes (39, 40) and at Φ_{GMO}=0 the lamellar L phase (FIG. 2, see Zheng et al.) termed lipoplexes. The intensity of DiO fluorescence as a function of time (FIG. 4B, see Zheng et al.) is significantly higher for LNPs comprising Φ_{GMO}=0.85 (blue data points) compared to Φ_{GMO}=0 (green data points). This is consistent with a considerably higher extent of fusion of endosomes with “nonlamellar” LNPs ( [symbol] _{II} /H_{II}) compared to regular “lipoplex” LNPs. Additional data are shown in 4 of Zheng et al.

The fact that [symbol] _{II}/H_{II} RNA-loaded LNPs fuse with isolated endosomes side-by-side on a glass slide is encouraging for their application as fusogenic LNPs for RNA delivery, but this is not representative of the fusion process in live cells where LNPs have to break out of an enclosing endosomal membrane. To evaluate how LNPs of different nanostructures fuse with the entrapping endosomal membranes in live cells, we conducted a fluorescence self-quenching experiment in live

HeLa cells (FIG. 4 C and D, see Zheng et al.). We employed two types of siRNA-loaded LNPs: cuboplexes ( [symbol] _{II}/H_{II} ) and lipoplexes (L) labeled with high molar percentages (1%) of a self-quenching dye, DiD. When the membranes of the LNP-siRNA complexes fuse with endosomes, DiD molecules will diffuse across the membrane, reducing the self-quenching of DiD and increasing its fluorescence. FIG. 4 C and D of Zheng et al. displays the obtained DiD fluorescence recovery at three time points (1, 3, and 5 h) after incubation with L (FIG. 4C, lipoplexes, see Zheng et al.) and [symbol] _{II}/H_{II} (FIG. 4D, cuboplexes, see Zheng et al.) siRNA-loaded LNPs. As time progresses, an increasing DiD signal (red fluorescence signal) is observed in cells treated with cuboplexes, indicating that indeed LNPs successfully fused with the endosomal membrane that internalized them. Comparatively at the same time point, cells treated with lipoplexes show little DiD fluorescence recovery, implying less effective endosomal membrane fusion. These results show that the nanostructure of LNP-RNA complexes plays a critical role in their ability to fuse and break out of endosomal compartments.

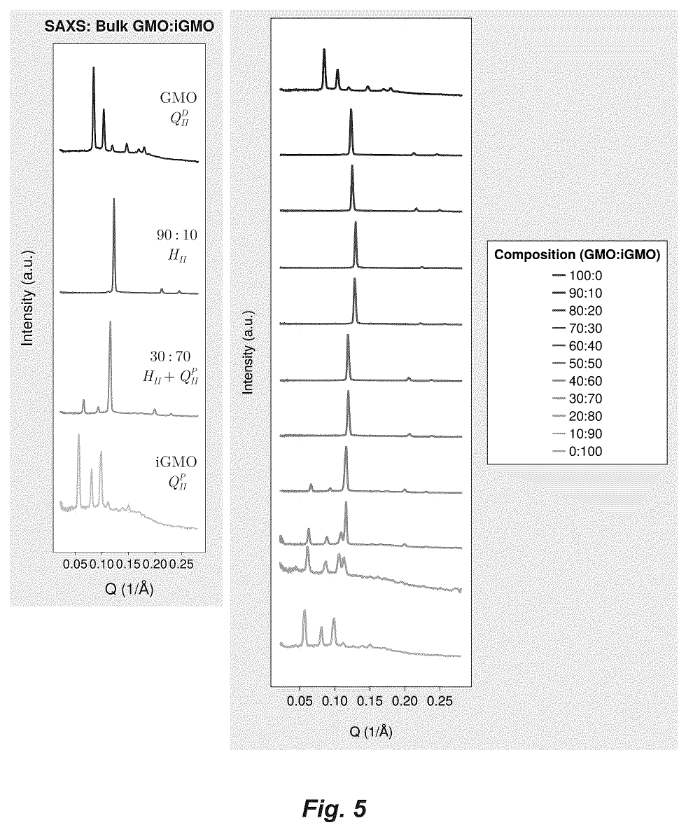

RNA Delivery of LNP-RNA Complexes of Different Nanostructures. We next investigated whether the ability for [symbol] _{II}/H_{II} RNA-loaded LNPs (cuboplexes) to efficiently fuse with endosomal membranes translates to better delivery of cargo and more efficient endosomal escape. Uptake of siRNA in HeLa cells and murine breast cancer (4T1) cells was quantified by flow cytometry (FIG. 5A, see Zheng et al.). To verify that the siRNA signal is not from siRNA outside the cell membrane or from dead cells, trypan blue (TB) was added to quench the fluorescence of siRNA outside the cellular membrane and that of membrane-compromised cells. The results show that HeLa cells treated with cuboplexes have significantly more cytoplasmic siRNA compared to cells treated with lipoplexes, and the siRNA uptake is comparable to that of Lipofectamine (LFA), a commercially available siRNA transfection agent. HeLa cells are well known to be relatively easy to transfect; however, in more resilient cell lines like 4T1 cells, we observe that cuboplexes continue to outperform lipoplexes in siRNA delivery capabilities.

To test the hypothesis that cuboplexes deliver more RNA to cells compared to lipoplexes because they more effectively fuse and break out of endosomal entrapment, we utilized live cell imaging to compare the cell internalization as well as endosomal entrapment of these two different LNP-RNA complexes (FIG. 5B, see Zheng et al.). DiO-labeled LNP-siRNA complexes (shown as red) were added to HeLa cells stained with Hoescht nuclei dye (blue) and imaged as a function of time (0, 30, 60, and 120 min). The higher amount of red signal around the nuclei would correspond to a higher extent of nanoparticle internalization (FIG. 5 C and E, see Zheng et al.). Lipoplexes show lower levels of internalization by HeLa cells compared to cuboplexes. To investigate the endosomal entrapment of the different LNP-siRNA complexes, early endosomes of HeLa cells were labeled with a fusion construct of Rab5a-GFP (shown as green). The cells were treated with LNP-siRNA complexes prepared with siRNA fluorescently labeled with Alexa Fluor 546 (AF546), shown as red, and monitored over time (5, 15, 30, and 60 min). When siRNA resides entrapped in the endosome, the fluorescent signal of siRNA and endosome would colocalize, appearing as yellow. Less colocalization, or yellow, would be an indication of less endosomal entrapment and more occurrences of successful endosomal escape. In FIG. 5 D and F of Zheng et al., compared to lipoplexes, cuboplexes showed less colocalization with the endosomes, indicating that less cuboplexes were entrapped.

Combined, lipoplexes show low levels of internalization and high amounts of siRNA-endosome colocalization (FIG. 5 C and D, see Zheng et al.) throughout the investigated timeline of up to 2 h (internalization) and 1 h (entrapment). This indicates that cuboplexes have superior ability not only to get internalized by cells at faster rates but also to efficiently evade endosomal entrapment and translocate RNA into the cytosol. We have shown that cuboplexes are more efficient at transfecting siRNA into cells compared to lipoplexes leading to better specific gene-knockdown performance. When replacing siRNA for mRNA, LNPs retain their structural identity (FIG. 2, see Zheng et al.) as well as their shape and size (Table 1). To show that cuboplexes containing siRNA silence genes more effectively than lipoplexes with siRNA, we performed an siRNA knockdown experiment with cuboplexes, lipoplexes, and LNPs formed using the state-of-the-art ionizable lipid formulations (IL-LNPs). The LNPs used for these transfection studies were prepared by nanoprecipitation in microfluidics, which is a well-established LNP preparation method. Dialysis was utilized to remove the solvent to minimize disruption to the LNPs. Other studies have shown that dialysis does have an impact on the structure of IL-LNPs. Specifically, changes in pH during dialysis drive IL-LNPs to have a more solid core, and the presence of ethanol disrupts the stability. However, in our case, LNPs use cationic lipid DOTAP, and there is no pH difference present during the dialysis process. Therefore, a structural change seen in IL-LNPs from dialysis is not observed.

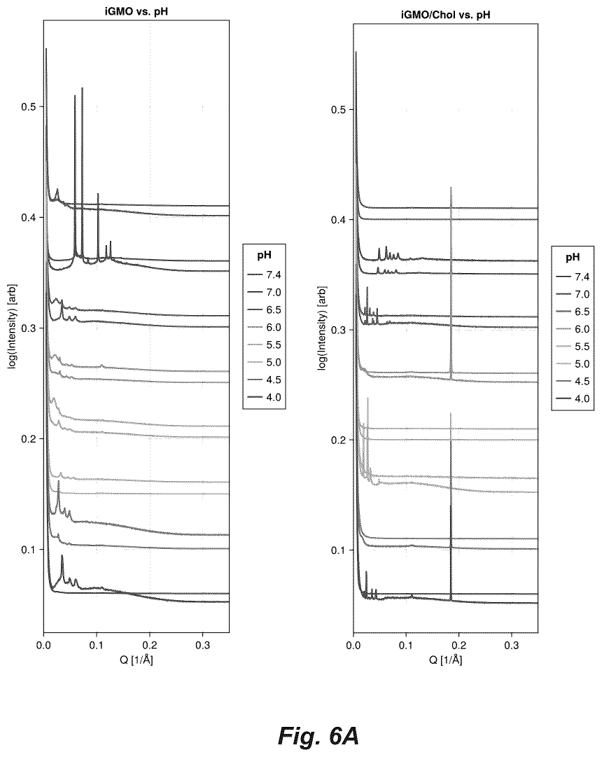

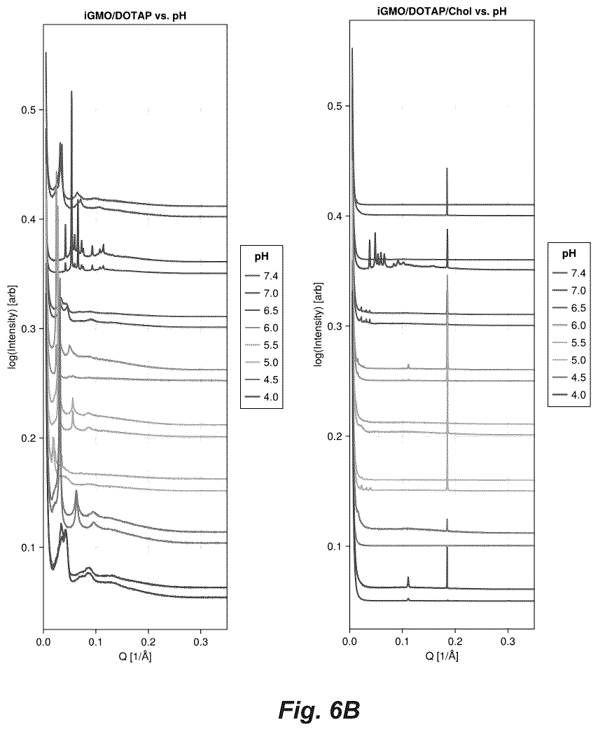

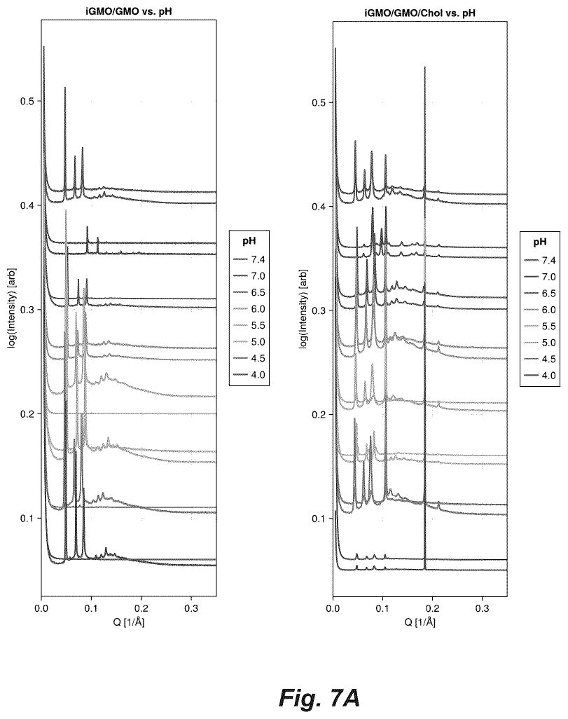

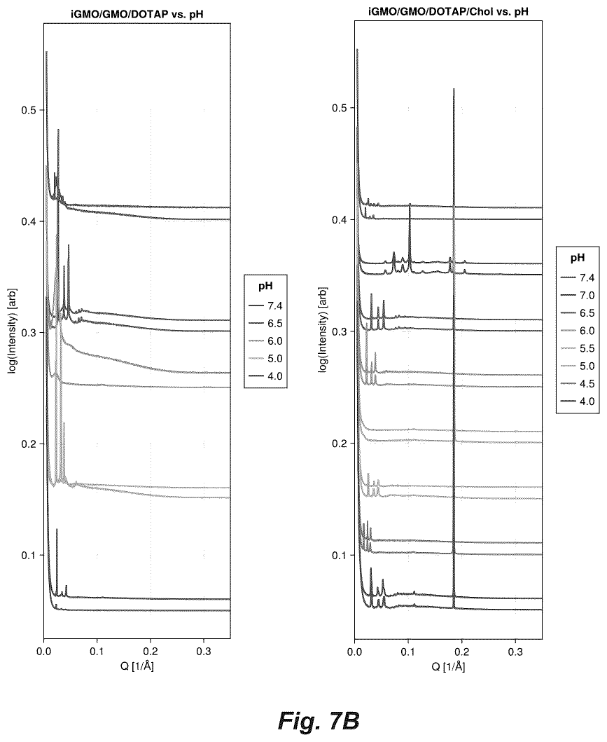

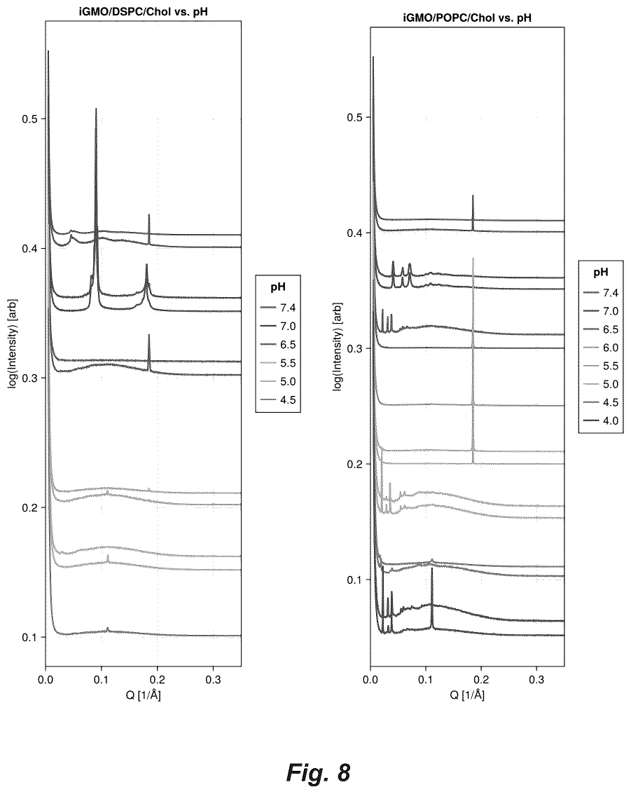

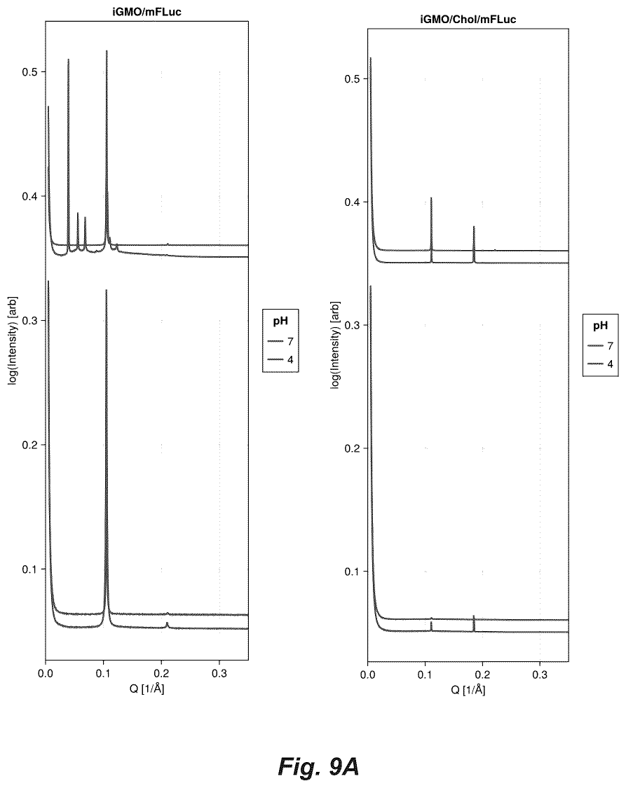

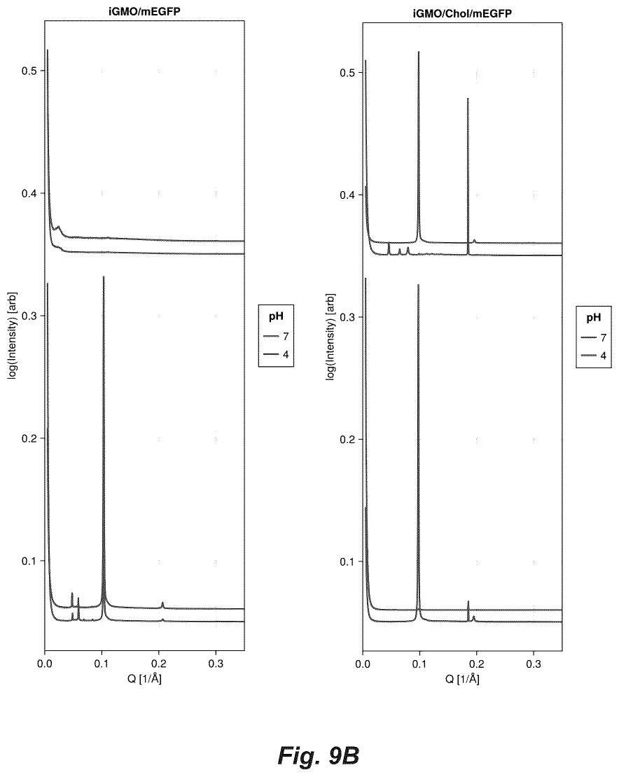

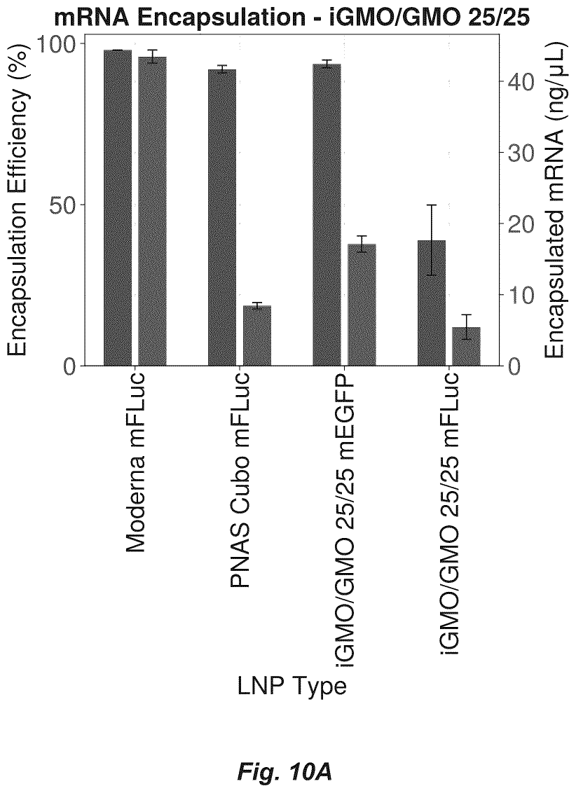

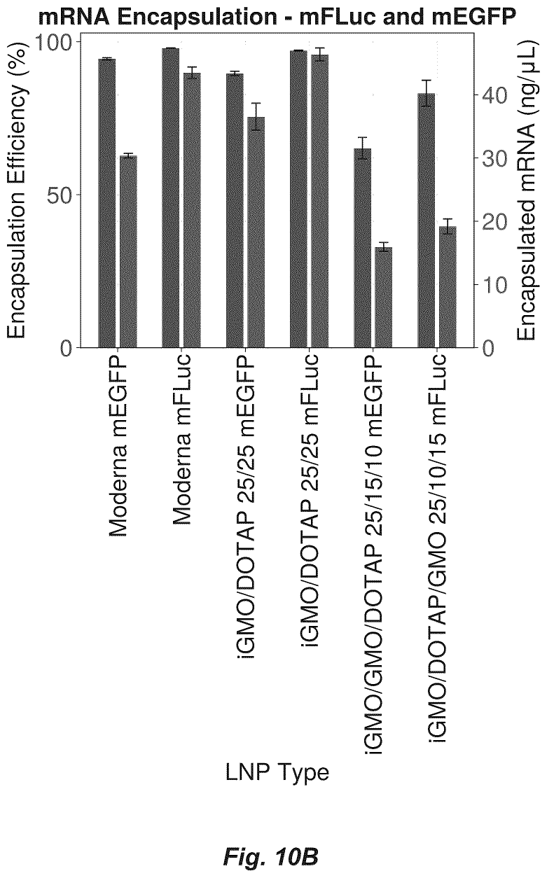

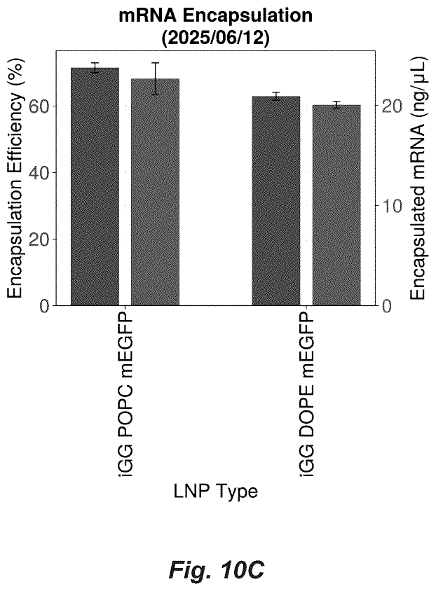

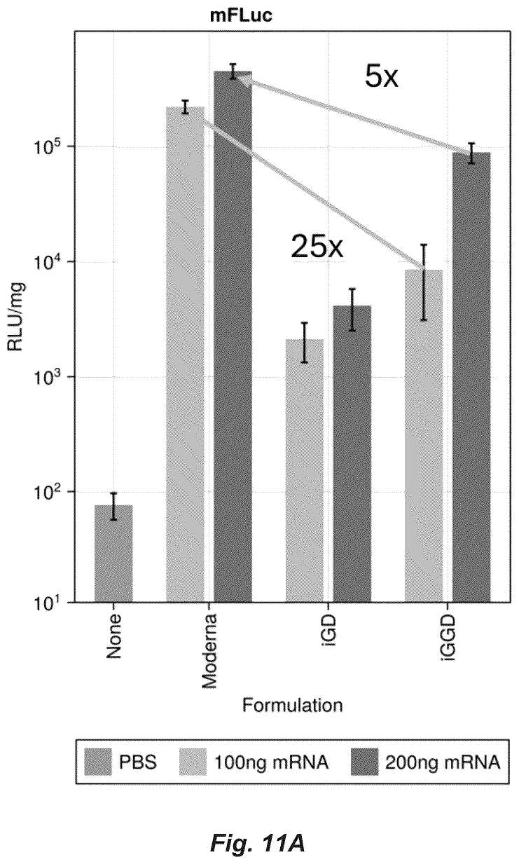

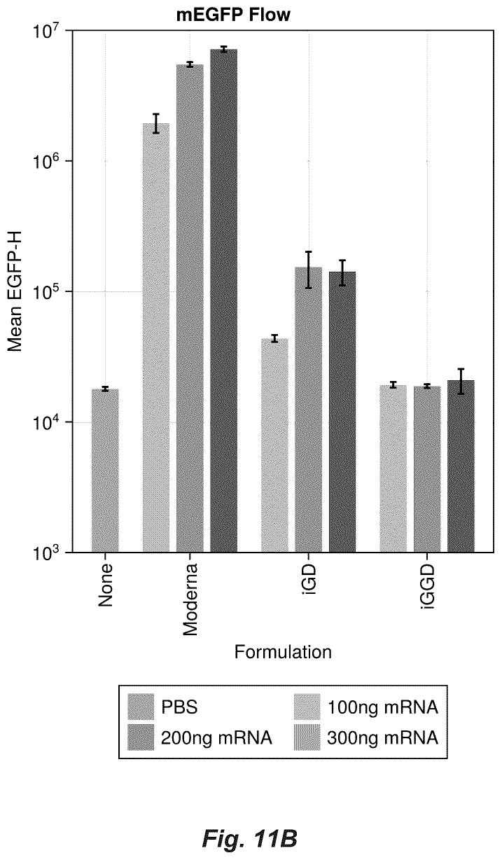

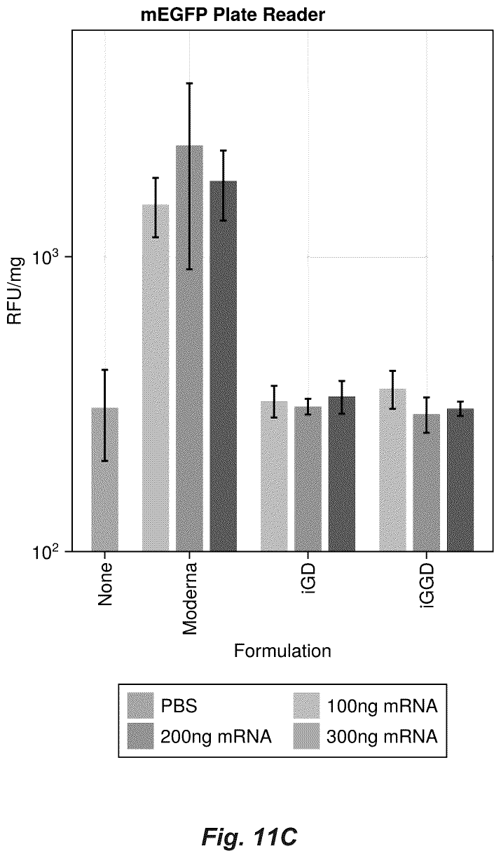

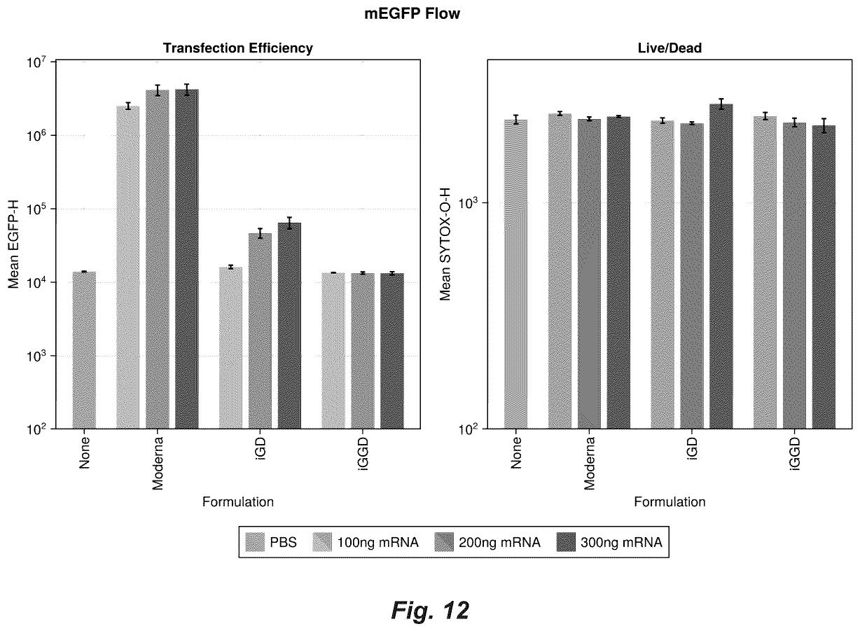

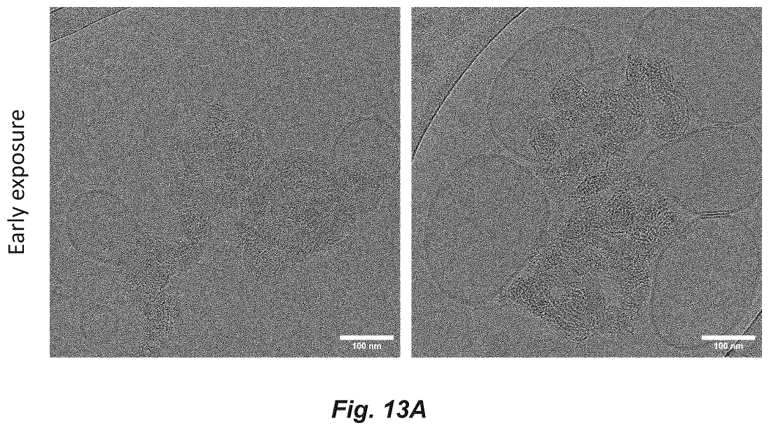

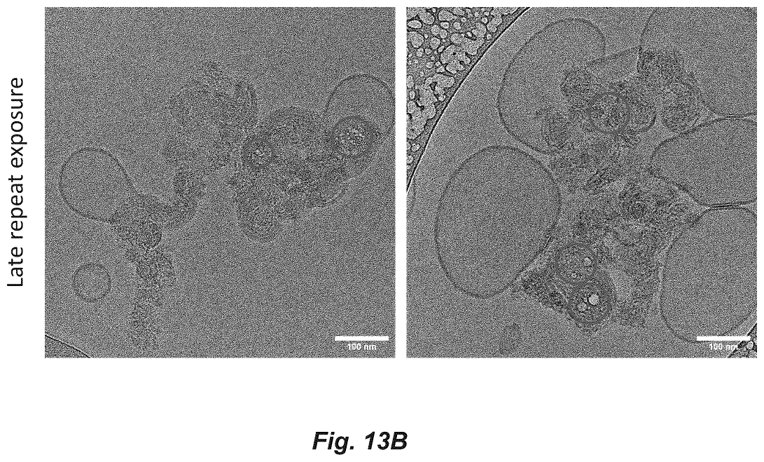

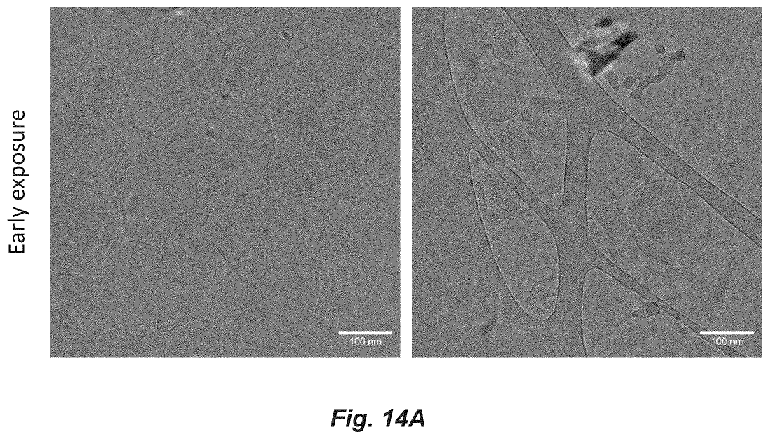

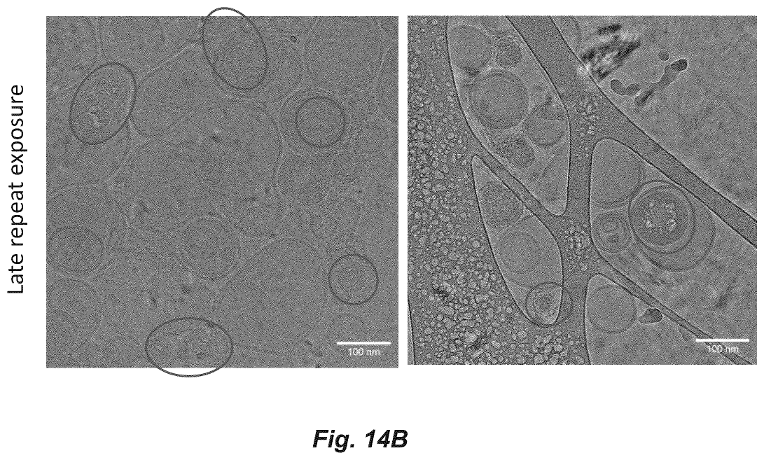

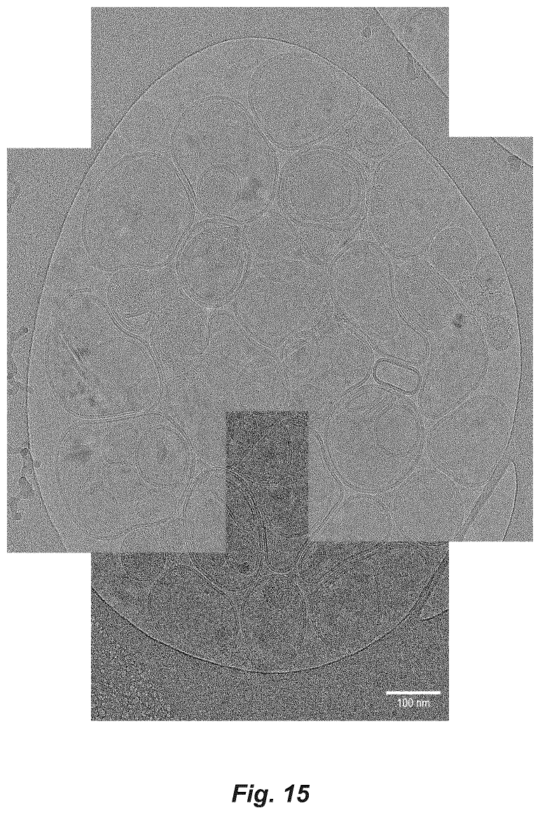

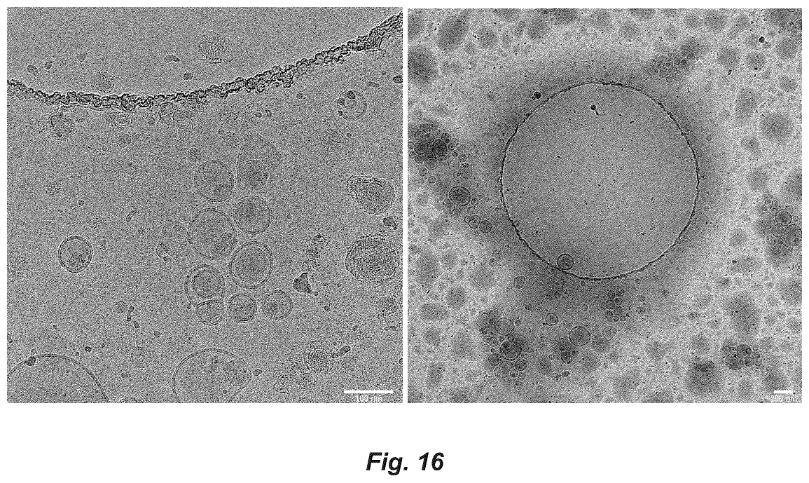

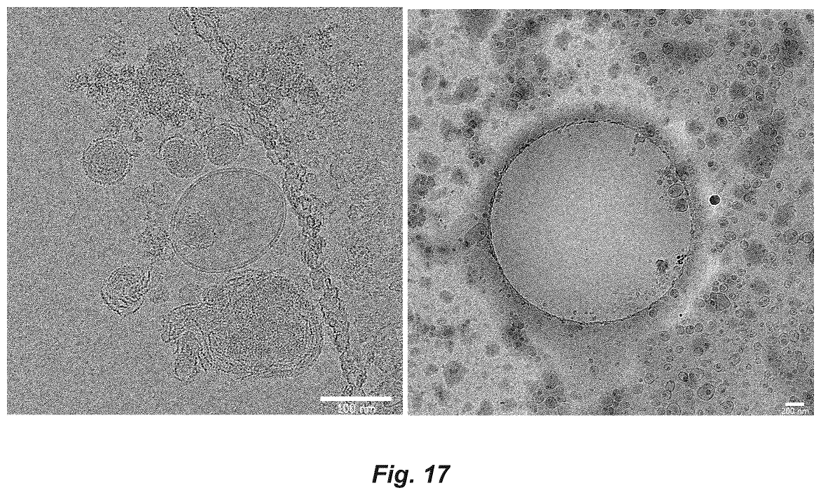

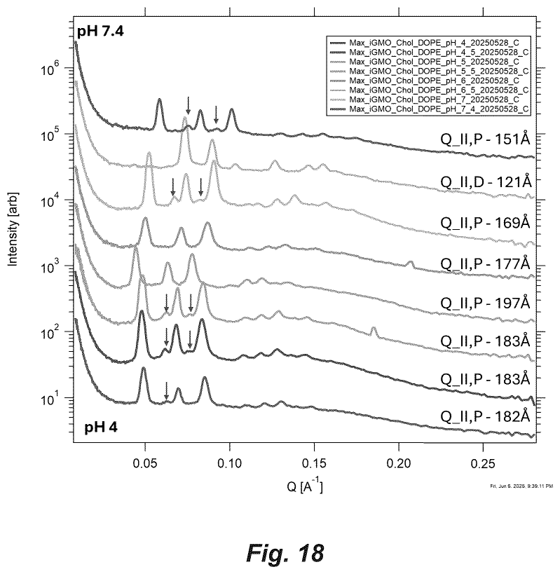

HeLa cells stably expressing green fluorescent protein (GFP) were treated with LNPs containing siRNA targeting GFP (siGFP) or scrambled siRNA (siNeg). LNP-siNeg samples were included as a negative control to test the effects of nonspecific gene knockdown. The level of GFP expression was evaluated by its fluorescence using flow cytometry. As shown in FIG. 6A of Zheng et al., cuboplexes successfully reduced GFP expression, while lipoplexes showed only minimal GFP silencing. IL-LNPs showed the strongest knockdown capabilities of all. This is consistent with our understanding of how ILs operate such that ILs become cationic inside the endosome electrostatically binding the endosomal membrane and facilitating fusion. To test how general the effect is with respect or other RNA-editing approaches, we employed cuboplex LNPs to deliver firefly luciferase-expressing mRNA (mFluc) to cells. It is clear that cuboplexes lead to more efficient activation of luciferase expression (FIG. 6B, see Zheng et al.) compared to parent lipoplex mRNA-LNPs. Both siRNA-mediated silencing and mRNA-mediated expression reveal that LNPs that are inherently more fusogenic (ILs due to electrostatic attraction and cuboplexes due to κ⁻ effects) are more efficacious. These data highlight the importance of designing not only the lipid identity and composition but also the overall nanostructure of the LNP.

To get further insight into the importance of LNP fusogenicity for efficient RNA delivery, we evaluated the approach of Sahay's lab that has demonstrated the beneficial effects of cholesterol on endosomal escape. We designed a GMO-LNP (with coexisting [symbol] _{II} /H_{II} phases) containing 30 mol % of cholesterol (GMO/DOPC: Chol: DOTAP: DOPE-PEG 55:30:14:1), which matches the composition utilized in state-of-the-art IL-LNPs. Previous studies have shown that cholesterol helps stabilize high-curvature membrane bud necks, and they should have a stabilizing effect also on membrane fusion pores. As schematically depicted in the inset of FIG. 6C of Zheng et al., the presence of fusion pores that are formed after merging two opposing membranes is fundamental to the endosomal escape process. It is expected that cholesterol from LNPs has the ability to stabilize high-curvature regions of the membrane fusion pores aiding in successful endosomal escape.

The transfection data show (FIG. 6C, see Zheng et al.) that for cells treated with complexes containing GMO and Chol (GMO/Chol-LNPs), a significant, more amount of luciferase is expressed compared to cells treated with complexes with DOPC and Chol (DOPC/Chol-LNPs). This is important because it reveals that cholesterol alone is not enough to elicit fusion. However, after the onset of membrane attachment enhanced by electrostatic effects in the case of ILs and κ⁻ in the case of GMO, cholesterol accumulation in the membrane pore would reduce the energetic cost of the topological transformation from endosomes encapsulating LNPs to a single endosome-LNP fused object. A lot more fusion pores are created when the cells are treated with GMO/Chol-LNPs or IL/Chol-LNPs, and membrane fusion plays an important role in the endosomal escape of LNPs. It is noteworthy that a plasma membrane integrity assay confirmed that all LNP-RNA complexes used in this study have negligible cytotoxicity (Fig. S4, see Zheng et al.). The possibility of replacing DOTAP with IL was also explored, and the immediate finding is that replacing cationic DOTAP with SM-102 ionizable lipids does not result in a well-ordered cuboplex (or any interesting nanostructure), and as expected, their performance in mRNA delivery and expression is quite poor compared to a cuboplex (Fig. S5, see Zheng et al.). These results suggest that a different ionizable lipid structure (not commercially available) would be needed to construct ionizable cuboplexes.

### Discussion.

We show that fusogenicity is a fundamental LNP property for efficacious RNA delivery and that LNP nanostructure identity is an additional handle to enhance it. Compared to lamellar structured LNP-RNA complexes, cubic and inverse hexagonal structured LNP-RNA complexes are able to fuse with endosomal membranes easier, hence leading to a higher extent of endosomal escape. These findings provide valuable insight in how nanostructures can affect nanoparticle-cell interactions, but also highlight the potential in utilizing structurally active lipids and nanoparticle structure as an additional handle to controlling the efficacy of drug delivery systems.

Endosomal escape has been a major hurdle in the development of successful RNA therapeutics. Our studies were able to show that by controlling the nanostructure of LNP-RNA complexes, we can influence the efficiency of endosomal escape without any aid from proteins, endosome acidification, or lipid ionization. Specifically, complexes with periodic bicontinuous cubic membrane interiors are able to promote membrane fusion between LNPs and endosomal membranes. When cholesterol is included, the process of membrane-fusion pore formation is facilitated. Cuboplexes have an intrinsic ability to lower the elastic cost of inducing membrane fusion followed by the topological transformation from an endosome with a nested LNP to a fused LNP-endosome state having a pore through which RNA can be transported into the cytosol. This enhancement in endosomal escape for cuboplexes led to better RNA delivery compared to its lipoplex counterparts.

These results show that in addition to a judicious choice of lipid composition, LNP nanostructure is a critical factor controlling fusogenicity. In other words, the inclusion of structurally active lipids is a potential design handle of nonviral delivery systems, and more research is required to fully unravel how nanoparticles of various structures interact with cells. Our studies highlight the importance of quantifying and predicting LNP fusogenicity. We show that LNP fusion ability cannot simply be justified by the effect of “inverted conical” lipids (negative spontaneous C₀<0) as bicontinuous cubic structures (C₀=0) are highly fusogenic. Rather than evaluating the ratio of bicontinuous cubic to hexagonal phases, we propose a more general description in that the Gaussian modulus, or rather the ratio between the Gaussian and the Bending moduli (κ̅ /κ), could be a better quantifier of LNP fusogenicity as it accounts for contributions of topology (generation of Gaussian curvature, e.g., during the formation of a fusion pore), elasticity, and C₀. However, unlike C₀ and κ that are experimentally accessible, κ̅ is hard to measure as it requires a controllable and reversible change of membrane topology. Nevertheless, SAXS methods have been successfully employed to quantify the Gaussian to bending moduli ratios of the monolayers interfacing with the water channels κ̅ₘ/κₘ in hexagonal and bicontinuous cubic phases.

For a ternary GMO/DOPC/DOPE (58:38:4 mol %) mixture, SAXS measurements yield κ̅ₘ/κₘ=−0.75 comparing to pure DOPE where κ̅ₘ/κₘ=−0.92 that is less fusogenic. In addition, as the importance of LNP nanostructure becomes more appreciated, further research needs to be done to fully explore how different preparation and purification methods might affect the nanostructure of LNPs beyond size and encapsulation efficiency.

### Conclusions.

Lipid nanoparticles (LNPs) are the most successful RNA delivery carriers to date and are used in FDA-approved products like the COVID-19 mRNA vaccine. Expanding LNP-based therapies hinges on efficient delivery to a variety of tissues and cells, a process often hindered by endosomal entrapment. It is well known that depending on lipid molecular properties, LNPs assemble into different nanostructures, but how these impart endosomal escape remains unknown. We demonstrated that combining lipid composition with nanostructure synergistically impacts the ability of LNPs to escape endosomes. LNP-RNA complexes prescribed with bicontinuous cubic and inverse hexagonal internal structures facilitate the topological transition of LNP-endosome fusion-pore formation. We showed that nanostructure is a potent handle to engineer highly efficient LNPs for RNA delivery.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1. Materials and Methods.

Materials. Glycerol monooleate (GMO) was purchased from Sigma-Aldrich (MO, USA). 8-[(2-hydroxyethyl) [6-oxo-6-(undecyloxy) hexyl]amino]-octanoic acid, 1-octylnonyl ester (SM-102) was purchased from Cayman Chemical Company (MI, USA). 1,2-dioleoyl-sn-glycero-3-phosphocholine (DOPC), 1,2-dioleoyl-3-trimethylammonium propane (DOTAP) and 1,2-dioleoyl-sn-glycero-3-phosphoethanolamine-N-[methoxy (polyethylene glycol)-2000] (DOPE-PEG), 1,2-distearoyl-sn-glycero-3-phosphocholine (DSPC), 1,2-dimyristoyl-rac-glycero-3-methoxypolyethylene glycol-2000 (DMG-PEG2000), and cholesterol (Chol) were purchased from Avanti Polar Lipids (AL, USA). siRNA targeting GFP and scrambled siRNA were purchased from ThermoFisher Scientific (IL, USA). Alexa Fluor 546 labeled siRNA (AF546-siRNA) was purchased from Qiagen (MD, USA). mRNA targeting firefly luciferase was purchased from TriLink Biotechnologies (CA, USA).

Preparation of LNPs and LNP-RNA Complexes. Lipid chloroform solutions were mixed at desired volumetric ratios. Chloroform was removed by treating the solution with a stream of nitrogen and then placing it under vacuum for at least 8 h. DOPE-PEG was dried in a separate vial. The dried lipid film was hydrated with sterile Milli-Q water and incubated overnight at 37° C. LNPs were obtained by sonicating the suspension with a cup horn system (Fisher Scientific) for 6 min at 100% amplitude. The temperature was maintained below 4° C. during sonication by a water chiller (Qsonica). The LNPs were transferred to the vial with dried DOPE-PEG and incubated for 1 h at 60° C. for postpegylation.

LNP-RNA complexes were formed by mixing LNPs with siRNA or mRNA at a charge ratio ρ(n_{DOTAP}/n_{NA}) of 3.

For transfection experiments, LNP-RNA complexes were formed by NanoAssemblr Ignite (Precision NanoSystems). Ionizable lipid (IL) LNPs were formed with a formulation of SM-102: DSPC: Chol: DMG-PEG2000 at molar ratios 50:10:38.5:1.5. Lipid chloroform solutions were mixed at desired volumetric ratios, and the solvent was removed as described above. The lipids were then dissolved in ethanol at a concentration of 10 mM. The total flow rate was maintained at 12 mL per min for all formulations. For cuboplexes, lipoplexes, GMO/Chol LNPs, and DOPC/Chol LNPs, 4:1 ratio of aqueous to ethanol phase was used, while a 3:1 ratio was used for formulating IL-LNPs. All formulations were made at a charge ratio p (n_{DOTAP} or IL/n_{NA}) of 6. Ethanol was removed by dialysis using Slide-A-Lyzer™ cassettes with a molecular weight cutoff of 3.5 k. The encapsulation efficiency was measured using QuantiFluor®RNA System (Promega).

Small-Angle X-Ray Scattering. LNP-RNA complexes (total lipid concentration 20 mM) were prepared and transferred to quartz capillaries (Hilgenberg Glas, Germany). Synchrotron SAXS was performed at beamline 12-ID-B of the Advanced Photon Source at Argonne National Laboratory. The average photon energy was 14 keV, and the data were radially averaged upon acquisition on a Pilatus 2M detector.

Cryogenic Electron Microscopy. To prepare samples for cryo-EM imaging, lacey carbon-coated 300 mesh copper grids (Electron Microscopy Sciences) were glow discharged at 15 mA for 30 s with the PELCO easiGLOW™ glow discharge system (Ted Pella). Then, 4 μL of sample was applied to the grids and incubated for 10 min. The grids were blotted with filter paper, and another 4 μL of sample was applied. The grids were then blotted for 2.5 s and plunge frozen in liquid ethane using Vitrobot Mark IV, under 4° C. and 100% humidity. The grids were kept in liquid nitrogen until imaging. Cryo-EM images were collected with Glacios Cryo-TEM (ThermoFisher) at 200 kV with a

Falcon 4 direct electron detector. Images were taken at −2 μm defocus to improve contrast. Fast Fourier transforms (FFTs) of images were obtained by ImageJ software.

Cell Culture. HeLa cells (ATCC), 4T1 cells (ATCC), HeLa-GFP cells (Cell BioLabs), and HeLa-Luc cells (Signosis) were cultured in full cell media consisting of Dulbecco's modified Eagle's medium (Corning), 10% fetal bovine serum (Gibco), and 1% penicillin-streptomycin (Gibco) at 37° C. with 5% of carbon dioxide.

Endosome Isolation and Characterization. Endosomes were isolated with the Trident endosome isolation kit (GeneTex). Particle concentration and size were measured by nanoparticle tracking analysis with NanoSight NS300 (Malvern Panalytical).

Membrane Fusion Studies. For confocal microscopy analysis, DiD (Biotum)-labeled LNPs were prepared by including 0.1% DiD in the lipid mixture. Endosomes were labeled by incubating with 20 μM NileRed for 1 h at 37° C. Free dye was removed from endosomes by centrifuging for 30 min at 10,000×g and discarding the supernatant. The endosomes were resuspended in PBS and incubated with DiD-labeled LNPs for 6 h at 37° C. The mixture was imaged on an LSM 800 (Carl Zeiss) confocal microscope.

For the FRET assay, endosomes were incubated with 20 μM DiO and 20 μM Dil for 1 h at 37° C. for colabeling. Free dye was removed, and endosomes were resuspended in PBS. Endosomes and lipid-siRNA complexes were incubated at 37° C. and measured for DiO fluorescence every 5 min. Fluorescence was detected with the Synergy Neo 2 microplate reader (Biotek).

Live Cell Imaging of the Uptake of LNP-RNA Complexes. Labeled LNP-siRNA complexes were prepared by including 0.1% DiI in the lipid mixture. HeLa-Luc were cultured on coverslip bottom dishes (ibidi) and stained with Hoescht dye. Complexes were added to the cells and imaged immediately on an LSM 800 (Carl Zeiss) confocal microscope: 405-nm laser was used for the Hoescht dye channel, and 488-nm laser was used for DiO and Dil. Images were acquired every 5 min for 3 h.

Cells were seeded onto poly-lysine-coated glass-bottom dishes at 1,000 cells/cm² density, and CellLight™ Early Endosomes-GFP reagent (ThermoFisher Scientific) was added after cell adherence. After 16 h of incubation, complexes with Alexa Fluor 546 labeled siRNA were added to the cells with a final siRNA concentration of 33 nM. The dishes were observed immediately under confocal microscopy. Images were acquired every 5 min for 4 h.

Flow Cytometry. HeLa cells (for siRNA uptake experiments) or HeLa-GFP cells (for siRNA transfection experiments) were seeded onto 12-well plates at 1,000 cells/cm² density prior to transfection. The following day, LNP-siRNA complexes were added to the cell media in which the cells were cultured in with a final siRNA concentration of 33 nM, and then incubated for 24 h. After incubation, the cells were trypsinized and washed with PBS. Cells were later suspended in FACS buffer (98% PBS, 2% FBS) and analyzed by flow cytometry (BD LSRFortessa X-20). For siRNA uptake experiments, the fluorescence of AF-546 was measured, and after the sample data were acquired, 0.4% trypan blue was added to the samples at a 1:10 ratio and analyzed by flow cytometry. For siRNA transfection experiments, the fluorescence of GFP was measured.

Luciferase Assay. Cells were seeded onto 96-well plates at 1,000 cells/cm² density prior to transfection. The following day, LNP-mRNA complexes were added to the cell media in which the cells are cultured in with 0.1 μg mRNA per well. The cells were incubated for 24 h, and the luciferase activity was evaluated by a luciferase assay (Promega). Luminescence was measured with the Synergy Neo 2 microplate reader (Biotek) and normalized by the cell protein mass of each well. Cell protein mass was quantified by the BCA assay (ThermoFisher Scientific).

### Example 2. Synthesis of iGMO and Characterization

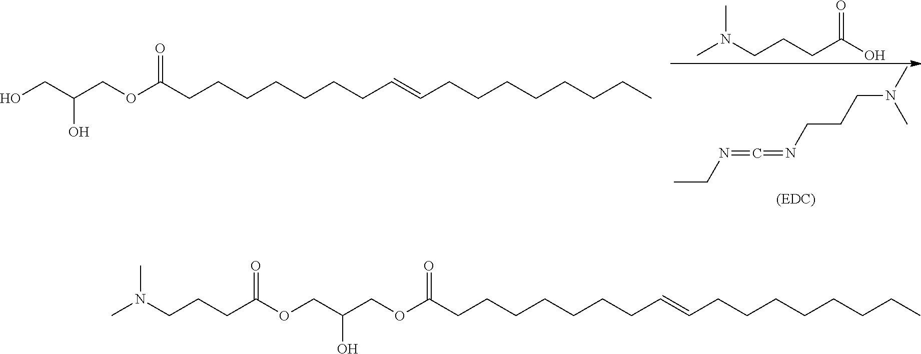

FIG. 1 characterizes iGMO by mass spectroscopy. FIG. 2 demonstrates the efficiency of an LNP comprising iGMO for delivering its cargo.

### Example 3. Composition of LNP and Characterization

Abbreviations: 8- [(2-hydroxyethyl) [6-oxo-6-(undecyloxy) hexyl]amino]-octanoic acid, 1-octylnonyl ester (SM-102); Cholesterol (Chol); 1,2-distearoyl-sn-glycero-3-phosphocholine (DSPC); 1,2-dimyristoyl-rac-glycero-3-methoxypolyethylene glycol-2000 (DMG-PEG2k); glycerol monooleate (GMO); ionizable GMO (iGMO); 1,2-dioleoyl-3-trimethylammonium-propane (chloride salt) (DOTAP); 1-palmitoyl-2-oleoyl-glycero-3-phosphocholine (POPC); 1,2-dioleoyl-an-glycero-3-phosphoethanolamine (DOPE);

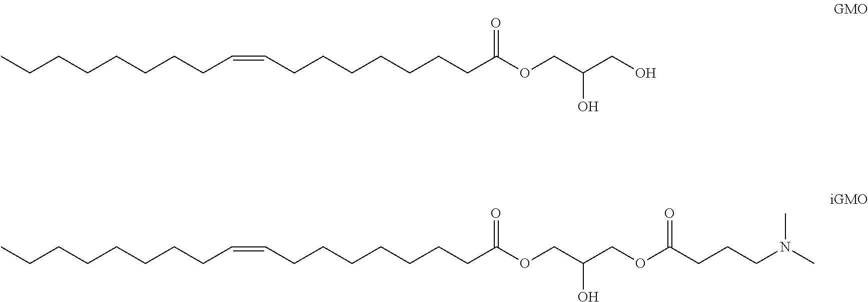 [SMILES: CCCCCCCC/C=C\CCCCCCCC(=O)OCC(O)CO ; CCCCCCCC/C=C\CCCCCCCC(=O)OCC(O)COC(=O)CCCN(C)C]

-ionizable amine having pKa of about 6.5

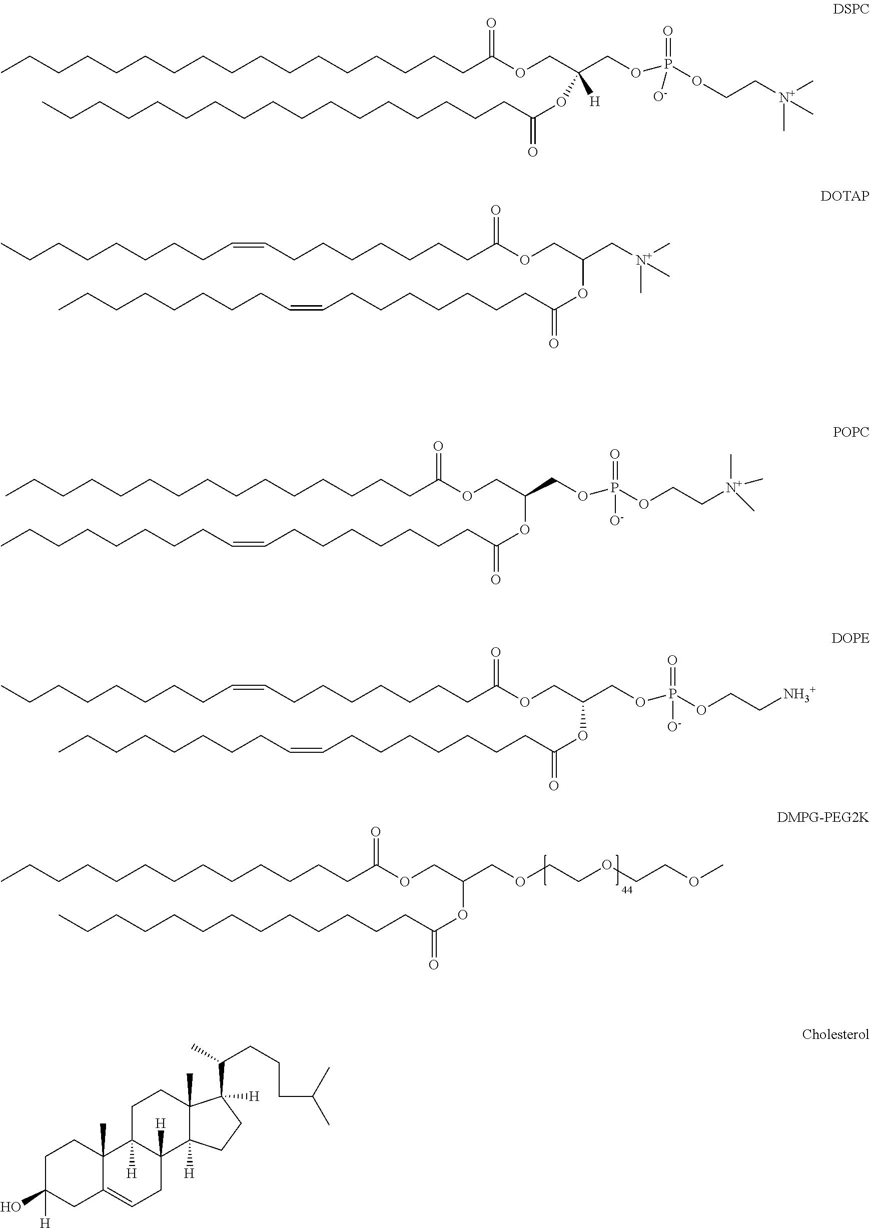

LNP comprises: iGMO 50 mol %, DSPC 10 mol %, Cholesterol 38.5 mol %, DMPG-PEG2k 1.5 mol %, and optionally comprising mRNA.

FIG. 3 shows iGMO LNP +/−mRNA size characterization. FIG. 4 shows iGMO LNP+mRNA formulation structural characterization.

### Example 4. Formulations prepared at N/P ratio=6 (unless otherwise noted).

### Base Formulation:

$$SM - 102 / Chol / DSPC / DMG - PEG 2 k 50 / 38.5 / 10 / 1.5 mol \% ( Moderna Spike Vax ) .$$

$$Control formulations :$$

$$Moderna Spike Vax ( Mod ) = SM - 102 / Chol / DSPC / DMG - PEG 2 k 50 / 38.5 / 10 / 1.5 mol \% .$$

$$PNAS Cuboplex ( Cubo ) = GMO / DOTAP / DMG - PEG 2 k 85 / 14 / 1 mol \% .$$

$$Experimental forulations :$$

$$iGMO LNP ( iG ) = i GMO / Chol / DSPC / DMG - PEG 2 k 50 / 38.5 / 10 / 1.5 mol \% .$$

$$iGMO / GMO LNP ( iGG ) = iGMO / GMO / Chol / DSPC / DMG - PEG 2 k 25 / 25 / 38.5 / 10 / 1.5 mol \% .$$

$$iGMO / DOTAP LNP ( iGD ) = i GMO / DOTAP / Chol / DSPC / DMG - PEG 2 k 25 / 25 / 38.5 / 10 / 1.5 mol \% .$$

$$iGMO / GMO / DOTAP LNP ( iGGD ) = iGMO / GMO / DOTAP / Chol / DSPC / DMG - PEG 2 k 25 / 15 / 38.5 / 10 / 1.5 mol \% .$$

$$iGMO / GMO + POPC LNP ( iGG - P ) = i GMO / GMO / Chol / POPC / DMG - PEG 2 k 25 / 25 / 38.5 / 10 / 1.5 mol \% .$$

$$iGMO / GMO + DOPE LNP ( iGG - D ) = i GMO / GMO / Chol / DOPE / DMG - PEG 2 k 25 / 25 / 38.5 / 10 / 1.5 mol \% .$$

### Example 5. Characterization of Formulations in Example 4.

### SAXS:

iGMO (and mixtures with GMO, DOTAP, Chol, mRNA) forms inverse hexagonal and cubic crystal structures associated with increased transfection efficiency, as well as lamellar phases.

iGMO undergoes pH-dependent phase transitions.

Replacing DSPC with POPC may favor inverse hexagonal phases across pH.

### DLS:

iGMO-based LNPs are slightly larger than other mRNA LNPs with comparable PDI.

Encapsulation efficiency- [symbol] uantifluor Assay:

iGMO-based LNPs encapsulate both mFLuc and mEGFP mRNA, in comparable encapsulated mRNA and encapsulation efficiency levels to control formulations.

mFLuc and mEGFP encapsulation show different trends in different formulations.

Dynamic Light Scattering (DLS): iGMO-based LNPs + mEGFP

| Formulation | Zavg (nm) | PDI |
|---|---|---|
| Moderna + mEGFP | 107.3 | 0.264 |
| PNAS Cubo + mEGFP | 102.4 | 0.137 |
| iGD + mEGFP | 142.7 | 0.278 |
| iGGD + mEGFP | 164.3 | 0.205 |
| iGG-POPC + mEGFP | 118.5 ± 0.35 | 0.365 ± 0.029 |
| iGG-DOPE + mEGFP | 133.8 ± 3.8 | 0.349 ± 0.028 |

iGMO-based LNPs have slightly larger diameters but PDIs are level when compared to control LNPs. The 100-200 nm range is considered optimal in literature. iGG-POPC/DOPE LNPs have suitable Zavg diameter, but with incread PDI has increased. Molar ratios can be adjusted to obtain a more monodisperse size distribution.

### In Vitro Transfection:

iGMO-based formulations deliver mRNA to cells with varying success—best case: iGGD+mFLuc (⅕ of Moderna+mFLuc).

Live/Dead stain shows minimal toxicity in line with control Moderna LNPs.

Differences between mFLuc and mEGFP possible.

### CryoEM:

Addition of GMO, DOTAP, or a combination thereof produces stable nanoparticles.

iGMO-based LNPs show outer bilayer and dense core components, with some empty bilayer and free cores as well.

mRNA is thought to reside in the dense cores.

X-ray scattering data for a mixture of iGMO, Chol, and DOPE shows increased preference for cubic structures when compared to mixtures with DSPC or POPC.

All publications, patents, and patent documents cited herein are incorporated by reference as though individually incorporated by reference. No limitations inconsistent with this disclosure are to be understood therefrom. The invention has been described with reference to various specific and preferred embodiments and techniques. However, many variations and modifications may be made while remaining within the spirit and scope of the invention.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

What is claimed is:

1. A compound of Formula I: 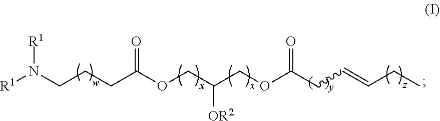
or a pharmaceutically acceptable salt thereof.
wherein
each R¹ is independently —(C₁-C₆)alkyl or H;
R² is H or —(C₁-C₆)alkyl;
w is 1-3 or 0;
each x is independently 1-3;
y is 7 or 1-10; and
z is 8 or 1-10.

2. The compound of claim 1, wherein each R¹ is methyl.

3. The compound of claim 1, wherein R² is H.

4. The compound of claim 1, wherein w is 1.

5. The compound of claim 1, wherein each x is 1.

6. The compound of claim 1, wherein each y is 7.

7. The compound of claim 1, wherein each z is 8.

8. The compound of claim 1, wherein the olefinic moiety of formula I has an E-configuration.

9. The compound of claim 1, wherein the compound is represented by Formula II: 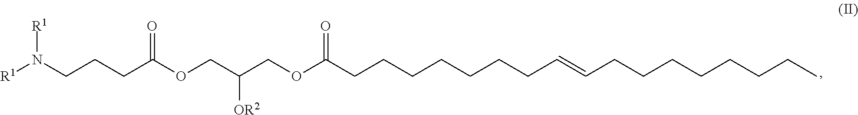
or a pharmaceutically acceptable salt thereof.

10. The compound of claim 1, wherein the compound is iGMO: 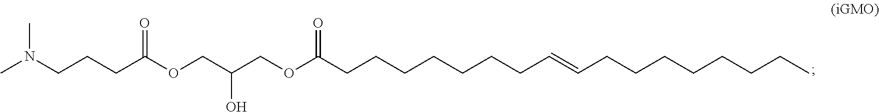 [SMILES: CCCCCCCC/C=C/CCCCCCCC(=O)OCC(O)COC(=O)CCCN(C)C]
or a pharmaceutically acceptable salt thereof.

11. A lipid nanoparticle composition comprising one or more lipids and one or more of a compound of claim 1.

12. The lipid nanoparticle composition of claim 11, further comprising RNA.

13. The lipid nanoparticle composition of claim 12, wherein the RNA is mRNA, siRNA, or both.

14. The lipid nanoparticle composition of claim 11, wherein the composition comprises:
about 45 mol % to about 55 mol % iGMO;
about 33.5 mol % to about 43.5 mol % cholesterol;
about 5 mol % to about 15 mol % 1,2-distearoyl-sn-glycero-3-phosphocholine (DSPC); and
about 0.5 mole % to about 2.0 mole % 1,2-dimyristoyl-rac-glycero-3-methoxypolyethylene glycol-2000 (DMG-PEG2k).

15. The lipid nanoparticle composition of claim 11, wherein the composition comprises:
about 20 mol % to about 30 mol % iGMO;
about 20 mol % to about 30 mol % glycerol monooleate (GMO);
about 33.5 mol % to about 43.5 mol % cholesterol;
about 5 mol % to about 15 mol % DSPC; and
about 0.5 mole % to about 2.0 mole % DMG-PEG2k.

16. The lipid nanoparticle composition of claim 11, wherein the composition comprises:
about 20 mol % to about 30 mol % iGMO;
about 20 mol % to about 30 mol % 1,2-dioleoyl-3-trimethylammonium propane (DOTAP);
about 33.5 mol % to about 43.5 mol % cholesterol;
about 5 mol % to about 15 mol % DSPC; and
about 0.5 mole % to about 2.0 mole % DMG-PEG2k.

17. The lipid nanoparticle composition of claim 11, wherein the composition comprises:
about 20 mol % to about 30 mol % iGMO;
about 10 mol % to about 20 mol % GMO;
about 5 mol % to about 15 mol % DOTAP;
about 33.5 mol % to about 43.5 mol % cholesterol;
about 5 mol % to about 15 mol % DSPC; and
about 0.5 mole % to about 2.0 mole % DMG-PEG2k.

18. The lipid nanoparticle composition of claim 11, wherein the composition comprises:
about 20 mol % to about 30 mol % iGMO;
about 20 mol % to about 30 mol % GMO;
about 33.5 mol % to about 43.5 mol % cholesterol;
about 5 mol % to about 15 mol % POPC or DOPE; and
about 0.5 mole % to about 2.0 mole % DMG-PEG2k.

19. A method for delivering biologically active cargo in a lipid nanoparticle into the cytosol of a cell comprising:
contacting a cell with a lipid nanoparticle wherein the lipid nanoparticle comprises biologically active cargo and a compound of claim 1;
wherein the lipid nanoparticle enters the cell by endocytosis to form an endosome, wherein the compound is ionized in the endosome's acidic environment to facilitate enhanced fusogenic disruption of the endosome's membrane and escape of the lipid nanoparticle from the endosome, thereby delivering biologically active cargo of the escaped lipid nanoparticle into the cell's cytosol.

20. The method of claim 19, wherein the biologically active cargo is mRNA, siRNA, or both.

21. The method of claim 19, wherein the compound is 3-((4-(dimethylamino)butanoyl) oxy)-2-hydroxypropyl (E)-octadec-9-enoate (iGMO).
